(19) United States
(12) Patent Application Publication
(10) Pub. No.: US 2024/0023585 A1
(43) Pub. Date: Jan. 25, 2024

(54) FOOD GRADE THICKENER AND METHODS FOR TREATING SWALLOWING DISORDERS

(71) Applicant: Trisco ICAP Pty Ltd, Carole Park, Queensland (AU)

(72) Inventors: Michael Tristram, Carole Park, Queensland (AU); Brenda Mossel, Carole Park, Queensland (AU); Santage Keighley, Carole Park, Queensland (AU)

(21) Appl. No.: 18/245,780

(22) PCT Filed: Sep. 28, 2021
(86) PCT No.: PCT/AU2021/051124

§ 371 Date: Mar. 17, 2023

(30) Foreign Application Priority Data
Sep. 28, 2020 (AU) 2020903490
Oct. 6, 2020 (AU) 2020903609

(51) Int. Cl. A23L 29/262; A23L 29/269; A23L 29/256; B65D 25/52; C08L 1/28; C08L 5/12; A61K 47/36; A61K 47/38; F16K 15/00

(52) U.S. Cl. CPC A23L 29/262; A23L 29/272; A23L 29/27; A23L 29/256; B65D 25/52; C08L 1/286; C08L 5/12; A61K 47/36; A61K 47/38; F16K 15/00

(57) ABSTRACT

The present invention provides a method for providing a food grade thickener, the method comprising the steps of: establishing an aqueous continuous phase of a first polysaccharide, adding a second polysaccharide to the continuous phase thereby forming a gelled mixture, hydrolysing the gelled mixture to reduce the viscosity of the gelled mixture, and adding a gum to the hydrolysed gelled mixture under conditions such that the gum only partially expresses its viscosity, thereby forming the food grade thickener. The invention also relates to a method of treating a subject suffering from a mastication and/or deglutition disease, disorder or condition, comprising the step of administering a foodstuff to the subject, wherein the foodstuff comprises the food grade thickener of the invention. The invention further relates to a storage and delivery system for a food grade thickener, comprising: a.) a container containing the food grade thickener of the invention, and b.) a pump dispenser sealingly attached to the container, wherein the dispenser comprises a valve for inhibiting or preventing drying of the composition in the container

### PRIORITY CLAIM

The present invention claims priority to Australian provisional patent application numbers 2020903490 and 2020903609, each of which are incorporated herein by reference in their entirety.

### FIELD OF THE INVENTION

The present invention relates to a food grade thickener. In particular the invention relates to a stable liquid food grade thickener composition comprising a thickening agent and a viscosity inhibitor composition, wherein the food grade thickener is suitable for increasing the viscosity of an aqueous liquid foodstuff. The food grade thickener maintains a relatively low viscosity in situ, and yet when diluted in an aqueous liquid or aqueous liquid solid mixture foodstuff the viscosity of the target foodstuff is increased significantly and in a relatively short period of time. Preferably the viscosity of food grade thickener of the invention is instantly released when the food grade thickener is diluted by allowing one or more of the components of the composition to fully express their viscosity-increasing effect. The present invention also provides use of the food grade thickener for the treatment or amelioration of or assistance with a swallowing disorder, such as dysphagia and/or odynophagia. Finally, a storage and delivery system is provided, which is a particularly convenient method for delivering the food grade thickener of the invention. However, it will be appreciated that the invention is not limited to these particular fields of use.

### BACKGROUND OF THE INVENTION

The following discussion of the prior art is provided to place the invention in an appropriate technical context and enable the advantages of it to be more fully understood. It should be appreciated, however, that any discussion of the prior art throughout the specification should not be considered as an express or implied admission that such prior art is widely known or forms part of the common general knowledge in the field.

In the food industry, it is often desirable to add viscosity enhancers to food formulations to increase the viscosity of the end product. Examples of such end products include yoghurts and desserts. It is also often desirable to provide viscous thickened liquids, particularly for the geriatric and convalescent markets. The thickened liquids need to be of a particular, known and repeatable viscosity to be applicable to these markets.

Currently, these products are pre-prepared by mixing the foodstuff with the viscosity enhancer before packaging, and providing the final thickened product already packaged. In some instances, the thickener is provided as a pre-mixed viscous emulsion or gel, which is then added to the food to be thickened, and stirred. It is often difficult to properly mix these gels into the food as the gel is not easily dispersed and tends to clump, resulting in lumpy end products.

Viscosity enhancers can also be provided in powdered form for the end user to mix into the food to be thickened. When these viscosity enhancers are dissolved into aqueous solution, they generally restrict the movement of water molecules and cause a considerable increase in viscosity, from thickening to gel formation. This may present problems with inconsistencies in incorporating the powder and has the potential to incorporate air into very thick products. Also, the addition of a powdered thickener usually results in a lumpy, inconsistent product which would not be safe or desirable for consumption, especially for geriatric consumers, or patients suffering from a mastication and/or deglutition disease, disorder or condition, such as dysphagia.

Viscosity enhancers may also be added to a foodstuff as an aqueous solution or the like. One of the problems with this method though is that the viscosity enhancer typically expresses its viscosity once in solution, making it difficult to dispense the solution unless the concentration of the viscosity enhancer is low. This has implications for the addition of the solution to the foodstuff, as the viscosity and concentration of the end product will be affected by the amount of solution added.

Furthermore, such solutions are typically not stable and must be used immediately or preservatised. Hence compositions of this type are typically not prepared, stored or sold in this form, but normally kept in powdered form until just prior to use in the production of the food product. Additionally, the dispensing of a powdered viscosity enhancer by volume, such as using a cup measure, is inherently inaccurate due to the variation in bulk density of the powders.

It is often desirable to provide viscous thickened liquids, particularly for the geriatric and convalescent markets. The thickened liquids need to be of a particular, known and repeatable viscosity to be applicable to these markets. Predetermined liquid viscosities have been developed by a number of regulatory bodies that are considered to have a clinically significant benefit in “slowing down” a dysphagia patient's swallow so that common co-morbidities of the disorder, such as aspiration pneumonia, are prevented. In light of the varying severity of swallowing disorders, the following professional guidelines are generally practiced clinically: mildly thick (nectar consistency); moderately thick (honey consistency); and extremely thick (pudding consistency). These guidelines typically correlate to 150, 400 and 900 mPa·s, respectively. These viscosity levels have now been included in a relatively new international framework called the IDDSI (International Dysphagia Diet Standardisation Initiative) Framework and described as Level 1—Slightly Thick, Level 2—Mildly Thick, Level 3—Moderately Thick, and Level 4—Extremely Thick. The IDDSI Framework not only describes the subjective attributes of the four consistency levels but also prescribes an objective test (the IDDSI Flow Test) with tightly defined ranges of measurement to ensure strict compliance with the consistency that is desired to be achieved. Noncompliance with these consistency parameters increase the risk of an unsafe swallow by a person with dysphagia that can lead to serious complications as mentioned above and could lead to death in frail and elderly patients.

In light of the foregoing, there exists a need for improved liquid food grade thickener compositions, which are stable and can be added in situ to any hot or cold foodstuff to give a thickened liquid foodstuff of known and repeatable viscosity, which may be used, for example, to feed subjects suffering from a mastication and/or deglutition disorder, such as dysphagia.

The present inventors are aware of other prior art documents in the field of this invention, such as JP 2007105018A, titled “Highly thickener-containing preparation” (i.e., JP2007), which relates to the preparation of thickener compositions which can be used to thicken a foodstuff.

JP2007 relates to the concept of providing a flowable thickener solution in which there is insufficient free water for dissolving a high-viscosity thickener (such as xanthan gum) so that it cannot express its full viscosity in situ, but can quickly express its viscosity when added to an aqueous target foodstuff. This is achieved by adding the high-viscosity thickener to an aqueous solution in which a low viscosity carboxymethylcellulose (CMC) and/or a low-viscosity alginate (for convenience, referred to herein as a “viscosity inhibitor”) is dissolved in water. By suppressing the dissolution of the high-viscosity thickener, the viscosity of the flowable thickener solution is kept low, and a thickened solution can be obtained when the flowable thickener solution is added to an aqueous target foodstuff. The flowable thickener solution disperses quickly in the target foodstuff and develops viscosity quickly.

In JP2007, the “highly viscous paste” (i.e., thickeners) disclosed are: xanthan gum, guar gum, locust bean gum, tara gum, tamarind gum, karaya gum, pectin, carrageenan, gellan gum, alginate, modified starch, and high viscosity CMC. The aqueous solution includes viscosity inhibitors that are low viscosity CMC (2-12 wt % preferably 4-10% by weight), or low viscosity alginate (2-12 wt % preferably 4-10% by weight).

JP2007 draws a distinction between a low viscosity CMC, as being less than 100 mPa·s, and a high viscosity CMC, with a 10% CMC aqueous solution having a viscosity of 1000 to 100000 mPa·s. Example 1 uses CMC having a viscosity of 18 mPa·s (10%, 20° C., B-type viscometer at 30 rpm), and Examples 2 to 5 use sodium alginate having a viscosity of 32.8 mPa·s (10%, 20° C., B-type viscometer 30 rpm). In these examples, when a high-viscosity thickener was added to an aqueous solution having a viscosity inhibitor, the resulting viscosities were in the range of 750 to 2,100 mPa·s, and when that flowable thickener solution was added to a target foodstuff in a 20:80 wt. % ratio, the viscosity was the range of 2,800 to 3,900 mPa·s.

The applicant has attempted to replicate the work disclosed in JP2007 and was surprised to find that it was not able to formulate a flowable thickener solution that is food grade, despite JP2007 asserting that the flowable thickener solution disclosed therein is a food grade thickener. Therefore, following the instructions of this prior art, the Applicant was not able to formulate a food grade flowable thickener solution that could assist in the treatment of a mastication and/or deglutition disease, disorder or condition. It was hypothesised by the Applicant that, despite JP2007 indicating that the resulting flowable thickener solution is food grade, in fact the viscosity inhibitor utilized in the examples in JP2007 is not food grade, and therefore there is no enabling disclosure of the provision of a food grade flowable thickener solution in JP2007. In particular, it is surmised by the Applicant that the inventors of JP2007 sourced industrial grades of viscosity inhibitors, not food grade viscosity inhibitors, especially as it is known that industrial grades of sodium CMC have lower apparent viscosities than food grades. This understanding seems to accord with the fact that no product has been commercialised by the Applicant of JP2007, suggesting that it did not meet the rigorous and stringent food grade standards required around the world for this kind of product.

To explain further, the Applicant sourced a range of food grade sodium CMCs, and found that the lowest molecular weight food grade that could be sourced (i.e., 10,000 Daltons) delivered a viscosity of 2000 cPs at 10% solution. In contrast, JP2007 describes a sodium CMC has a viscosity of 18 cPs for a 10% solution. This is a significant contributing factor to the differences that the Applicant has uncovered between JP2007 and the present invention, as will be explained further below. It is also well known that the viscosity of a CMC solution increases rapidly with concentration. A “rule of thumb” is that viscosity increases 8 to 10-fold when the concentration is doubled, and therefore a viscosity of 18 cPs at a 10% solution is not feasible. Using the lowest viscosity food grade sodium CMC available in the method of JP2007 resulted in viscosities that are too high to be flowable or pumpable, and will not readily disperse in a target foodstuff. Further comparative experimental data can be seen in the experimental section below. For completeness, it is worth noting that the Applicant utilized a Silverson mixer with a general-purpose stator, which is considered to be equivalent to the “Dispermix” disclosed in JP2007, and therefore the differences the Applicant have found with this prior art are not due merely to the mixing conditions or equipment used to prepare the formations discussed herein.

In summary, and as shown further in the experimental section below, when food grade ingredients are used in the teachings of JP2007, the composition produced is not flowable, pumpable and/or dispersible in an aqueous foodstuff. Therefore, the compositions of JP2007 are comprised of ingredients that are not food grade and hence are not appropriate in the treatment of a subject suffering from a swallowing disorder. Despite what is asserted in JP2007, no food grade thickener has been produced in that document. The present invention is an advance over the prior art because the Applicant has been able to prepare a thickener solution that is flowable, pumpable, that readily disperses in a target aqueous foodstuff, and that is food grade, and therefore that can assist in the treatment of a subject suffering a swallowing disorder.

What is needed is a methodology to produce a food grade thickener (using food grade ingredients) that addresses one or more of the objectives of the invention below. As disclosed herein, the Applicant has been able to formulate a food grade thickener that presents a patentable improvement over JP2007.

an object of the present invention to overcome or ameliorate one or more the disadvantages of the prior art, or at least to provide a useful alternative.

It is an object of a preferred embodiment of the present invention to provide a food grade thickener solution that is flowable, and pumpable, and is dispersible into an aqueous liquid so that the thickened liquid is homogenous, and is food grade.

It is an object of further preferred embodiments of the invention to provide a food grade

thickener that also has one or more of the following advantages: is homogenous (i.e., does not contain lumps or domains of undispersed thickener), has sufficient speed of hydration so the peak viscosity is reached within a short time frame (i.e., around 30-60 seconds) at low shear (i.e., 30-80 BPM with a fork/spoon), has ability to withstand shear on delivery with a food grade pump, does not separate or shows no substantial separation over time, is clear/transparent (i.e., imparts little or no colour to the target foodstuff), and has little or no odour and/or is sufficiently highly concentrated so that a relatively small addition can safely modify the texture without impairing the flavour or other desired attributes of the target foodstuff to be thickened.

### SUMMARY OF THE INVENTION

According to a first aspect, the present invention provides a method for providing a food grade thickener, the method comprising the steps of:
providing an aqueous phase,
adding a polysaccharide to the aqueous phase thereby forming a gelled mixture,
hydrolysing the gelled mixture to reduce the viscosity of the gelled mixture, and adding a gum to the hydrolysed gelled mixture under conditions such that the gum only partially expresses its viscosity, thereby forming the food grade thickener.

In one embodiment, the aqueous phase comprises water and a polysaccharide (i.e., the first and/or second polysaccharide as discussed below) is then added to that aqueous phase. In another embodiment, an aqueous continuous phase is firstly established with a first polysaccharide, to which is added a second polysaccharide to thereby form the gelled mixture.

The invention according to the first aspect essentially comprises a viscosity inhibitor composition into which is added a thickening agent in the form of a gum, wherein the viscosity inhibitor composition is formulated such that the gum only partially expresses its viscosity.

The present invention is a significant advance over the prior art. In some embodiments, in an initial step, the invention provides for an aqueous continuous phase of a first polysaccharide, and then a gelled mixture is formed within that aqueous continuous phase using a second polysaccharide. The gelled aqueous continuous phase is then hydrolysed to obtain a predetermined viscosity. The hydrolysis conditions are preferably around 80-90° C. for a period of less than 20 hours, but can be shorter or longer than this preferred time, and/or at higher or lower temperatures than this preferred temperature range. Preferably one or more of the steps of the method are conducted under low shear conditions, which is in contrast to the prior art which uses high shear conditions, as will be explained further below. In a final step, a sufficient quantity of gum, such as xanthan gum, is added to the hydrolysed continuous phase at a concentration preferably around 4 to 8 wt. % to produce a suspension or dispersion of xanthan gum in the hydrolysed continuous phase to produce an apparent viscosity of around 4,000 cP measured at 20° C. using a Brookfield viscometer #3 spindle at 5 RPM. The food grade thickener should preferably resist separation of the components of the composition, and yet retain thixotropy and resist shear deformation so as to be dispensable by a delivery pump and retain the ability to rapidly thicken a target foodstuff.

The Applicant has found that the inventive method disclosed herein provides control over what are understood to be the “cohesion” and “adhesion” of the thickener system, which are related to the pumpability and pourability of the food grade thickener of the invention. The “cohesion” and “adhesion” parameters are measured by the following tests:
cohesion—an apparent viscosity of about less than about 5,000 cPs measured at 20° C. using a Brookfield viscometer #3 spindle at 5 RPM , and

adhesion—a resistance to flow of greater than about 15 cm at 20° C. at 30 seconds measured using a Bostwick consistometer.

It will be appreciated that other methods could be used to measure the “cohesion” and “adhesion” parameters of the system, or equivalents thereof.

It has been found that viscosity measurements alone do not provide a sufficient characterisation of the system under study. However, preparing a food grade thickener of the invention that meets these criteria is flowable, and pumpable, and is dispersible into an aqueous foodstuff. The food grade thickener of the invention is also homogeneous, and does not impart any additional inhomogeneity to the aqueous foodstuff into which it is combined. Without wishing to be bound by any theory, it is believed that the second polysaccharide is initially “tempered” by the method and has a tertiary structure to accommodate the gum and allow it to hydrate only slightly, and such that the desired cohesion/adhesion properties are achieved. Utilising a combination of a first polysaccharide to initially prepare a continuous phase, into which is dispersed a second polysaccharide forms a gelled network, and then hydrolysing the combined polysaccharide gelled network seems to break down the chemical bonds of network in such way so as to allow the gum to only partially hydrate once added, thereby inhibiting its viscosity expression.

It has been found by the Applicant that the steps of, first, establishing an aqueous continuous phase of a first polysaccharide, and then second, adding a second polysaccharide to the continuous phase thereby forming a gelled mixture, and then hydrolysing the gelled mixture to reduce the viscosity of the gelled mixture to a predetermined range, enables the food grade thickener of the invention (i.e., including the gum) to have one or more of the following advantageous properties, and in particularly preferred embodiments a plurality of these embodiments, and in some preferred embodiments all of the following properties: homogenous (i.e., does not contain lumps or domains of undispersed thickener), has sufficient speed of hydration so the peak viscosity is reached within a short time frame (i.e., around 30-60 seconds) at low shear (i.e., 30-150 BPM with a fork/spoon), has ability to withstand shear on delivery with a food grade pump (i.e., is a non-Newtonian/thixotropic fluid), does not separate over time, is clear/transparent (i.e., little or no colour) in the target foodstuff, and has no odour and/or taste so as to not impart any flavour to the target foodstuff to be thickened. The particular steps of the invention are not foreshadowed or even hinted at by JP2007, and enable a food grade thickener to be produced which cannot be produced by this prior art document. The food grade thickener of the invention is an advance over commercially available products for at least the reasons described above.

As will be shown below, in contrast to the inventive thickener composition described herein, the prior art does not display an apparent viscosity of less than 5000 cPs, which is a proxy for preferred cohesion properties, and which is related to dispersibility. Additionally, the prior art does not display a resistance to flow of greater than about 15cm at 20° C. at 30 seconds measured using a Bostwick consistometer, which is a proxy for preferred adhesion properties, which are related to pumpability.

### First Polysaccharide

In some embodiments, in a first step of the method of the invention an aqueous continuous phase of a first polysaccharide is established. The first polysaccharide may be a single polysaccharide or a plurality of polysaccharides. The first polysaccharide may be selected from the group consisting of agar, alginic acid, carrageenan, guar gum, gum tragacanth, gum ghatti, microcrystalline cellulose, sodium carboxymethylcellulose, methyl cellulose, hydroxypropylmethylcellulose, hydroxyproylcellulose, methylethylcellulose, gum karaya, locust bean gum, tara gum, psyllium seed gum, quince seed gum, a pectin, furcellaran, gellan gum, konjac, sodium alginate, xanthan gum and any combination thereof. It will be appreciated that, whatever first polysaccharide is utilised, it is food grade. It will be appreciated that some polysaccharides may be modified prior to use, for example, they may be modified to have a higher or lower molecular weight, or to have a different viscosity. In some embodiments, the polysaccharides may also be modified to adjust the gelling temperature. In some embodiments, the gelling temperature is adjusted to be between to 100° C. or 20 to 100° C. or 30 to 50° C. or 40 to 60° C. or 50 to 70° C. or 60 to 80° C. or to 90° C. or 80 to 95° C. or 90 to 100° C. In one embodiment, the gelling temperature could be modified through the addition of gelling cations. In some embodiments the polysaccharides may be modified to have a lower or higher acylation, or be used in a low acyl or high acyl form. It will appreciated that low acyl refers to polysaccharides that have been partly (degree of acylation 1 to 50%) or fully deacylated (degree of acylation less than 1%) and high acyl refers to polysaccharides that contain a relatively high number of acyl substituents, for example, a degree of acylation greater than 50% acylation.

To establish the aqueous continuous phase, in preferred embodiments the pH of water is adjusted to pH 3-4 with a food grade acidifier. Any food grade acidifier may be used, but in one embodiment glucono-delta-lactone (GDL) is used. The first polysaccharide is then added to the acidified water and mixed to incorporate, preferably under high shear conditions. In preferred embodiments the concentration of the first polysaccharide is wt. %, or 0.01 to 0.06 wt. % or in a ratio of 1:50 to 1:100. However, it will be appreciated that other concentrations may be used, such as 0.002, 0.004, 0.006, 0.008, 0.012, 0.014, 0.016, 0.018, 0.02, 0.04, 0.06, 0.08, 0.10, 0.2, 0.4, 0.6, 0.8, of 1.0 wt. %. It will also be appreciated that the concentration used in this step may vary based on the particular first polysaccharide that is used, and the concentration of the gelling cation.

In some embodiments, once the first polysaccharide has been dispersed in acidified water, a gelling cation may be added and optionally one or more preservatives. A suitable gelling cation is CaCl₂ at 0.001 wt %. However, it will be appreciated that other gelling cations may be used and at different concentrations. Use of a gelling cation may provide additional stability to the final food grade thickener, and may assist in avoiding separation of the food grade thickener over time. A suitable preservative is potassium sorbate that may be incorporated at around 1000 ppm. However, it will be appreciated that other preservatives may be used and at different concentrations.

Preferably, the solution is then heated to “melt” the first polysaccharide and to establish the aqueous continuous phase comprising the first polysaccharide. In some embodiments the solution is heated to the setting temperature of the first polysaccharide. In some embodiments the solution is heated to the melting temperature of the first polysaccharide. In some embodiments the solution is heated to between the setting and melting temperatures. In other embodiments, the first polysaccharide is heated to well above either of the setting and melting temperatures. In preferred embodiments, the solution is heated to 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 99.5 ° C. It will be appreciated that divalent cations are more efficient in promoting the gelation of some polysaccharides compared to monovalent ions. It will also be appreciated that the gelling and setting temperatures of some polysaccharides may increase with cation concentration.

Second Polysaccharide

In a further step of the method of the invention, a second polysaccharide is added to the continuous phase to thereby form a gelled mixture. In some embodiments of the invention, the second polysaccharide comprises one or more materials selected from the group consisting of agar, alginic acid, carrageenan, guar gum, gum tragacanth, gum ghatti, microcrystalline cellulose, sodium carboxymethylcellulose, methyl cellulose, hydroxypropylmethylcellulose, hydroxyproylcellulose, methylethylcellulose, gum karaya, locust bean gum, tara gum, psyllium seed gum, quince seed gum, a pectin, furcellaran, gellan gum, konjac, sodium alginate, and xanthan gum. In some embodiments of the invention, the second polysaccharide comprises one or more materials selected from the group consisting of microcrystalline cellulose, sodium carboxymethylcellulose, methyl cellulose, hydroxypropylmethylcellulose, hydroxyproylcellulose, methylethylcellulose.

In some preferred embodiments of the invention, the second polysaccharide is sodium carboxymethylcellulose (CMC). In some preferred embodiments of the invention, the second polysaccharide is a low molecular weight sodium CMC. Preferred molecular weights are in the range of 5,000 to 10,000, 10,000 to 15,000, 15,000 to 20,000, 20,000 to 25,000, or 25,000 to 30,000 Daltons. In some preferred embodiments, the second polysaccharide is a mixture of low molecular weight sodium CMCs. In this embodiment of the invention, the first sodium CMC and the second sodium CMC are used in a ratio of about 1:1. However, it will be appreciated that other ratios may be suitable, for example 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10. It will be appreciated that the first and second sodium CMC are food grade.

In some embodiments of the invention, a sufficient amount of the second polysaccharide is added to the aqueous continuous phase to obtain a target concentration of about 3 to 5 wt. %. However, it will be appreciated that a range of concentrations may be used, such as 0.5%, 1% , 2% , 3% , 4% , 5% , 6% , 7% , 8% , 9% , 10%, 11% , 12% , 13% , 14% , 15% , 16% , 17% , 18% , 19% 20%, 21% , 22% , 23% , 24% , 25%, 26%, 27%, 28%, 29% or 30% or any other concentration in between. It will also be appreciated that the concentration used in this step may vary based on the particular polysaccharide that is used.

The products in the market place are understood to not use combinations of polysaccharides that have differing properties. The Applicant has found that using a combination of polysaccharides may enable fine tuning of the properties of the gelled mixture so as to achieve a final target concentrate having the desired apparent viscosity (Brookfield) and resistance to flow (Bostwick), as well as pumpability and characteristics such as clarity and dispersibility. It is known that the molecular weight and the degree of substitution (DOS) of the polysaccharide molecule has a major influence on the solution characteristics. Generally speaking, as the DOS increases, the clarity of the solution increases, as does the stability, however the interactions (via hydrogen bonding) and the thixotropy can reduce. There are also competing effects due to the molecular weight.

It would be ideal to obtain advantageous properties from both high molecular weight forms of polysaccharides, and low molecular weight forms of polysaccharides. For example, the stability and clarity that tends to come from the use of a high molecular weight polysaccharide is preferable, and an almost Newtonian behaviour (thixotropy) of the continuous phase is preferable, which tends to be a characteristic of low molecular weight forms of polysaccharides. The almost Newtonian behaviour is preferable otherwise it is difficult to introduce sufficient gum and also achieve the properties of pumpability, flowability, dispersibility, and stability over time. When the food grade thickener experiences shear during a pumping method, the viscosity (resistance to flow) will decrease, which may affect the gum, consequently affecting the time it may take to express its viscosity when added to a target foodstuff, and negatively impacting the utility of the food grade thickener of the invention. A preferred objective of the food grade thickener of the invention is to provide an International Dysphagia Diet Standardisation Initiative (IDDSI) of <30 seconds. The IDDSI Framework consists of a continuum of 8 levels (0-7). Levels are identified by numbers, text labels and colour codes. (see https://iddsi.org/ for further information and detail on the IDDSI framework).

As used herein, the term polysaccharide includes synthetic polysaccharides, naturally occurring polysaccharides, a polysaccharide fragment, and any combination thereof. The synthetic polysaccharide is selected from the group consisting of microcrystalline cellulose, sodium carboxymethylcellulose, methyl cellulose, hydroxypropylmethylcellulose, hydroxypropylcellulose, methylethylcellulose, polyvinylpyrrolidone (PVP), a carboxyvinyl polymer, a methyl vinyl ether, a maleic anhydride polymer, an ethylene oxide polymer and any combination thereof. The naturally occurring polysaccharide is selected from the group consisting of scleroglucan, dextran, elsinan, levan, alternan, inulin, a gluco-oligosaccharide, an Acacia tree polysaccharide extract, an arabinoxylan, curdlan, a Larix occidentalis polysaccharide extract, a Larix laricina polysaccharide extract, a Larix decidua polysaccharide extract, a Larix sibirica polysaccharide extract and any combination thereof. The polysaccharide fragment is selected from the group consisting of a pullulan fragment, a soy bean polysaccharide fragment, an arabinogalactan fragment, a gum arabic fragment, an agar fragment, an alginic acid fragment, a carrageenan fragment, a guar gum fragment, a gum tragacanth fragment, a gum ghatti fragment, a gum karaya fragment, a locust bean gum fragment, a tara gum fragment, a psyllium seed gum fragment, a quince seed gum fragment, a pectin gum fragment, a furcellaran fragment, a gellan gum fragment, a konjac fragment, a sodium carboxymethylcellulose fragment, a sodium alginate fragment, a xanthan gum fragment and any combination thereof. Also falling within the scope of the invention is the use of an ionic polysaccharide which is selected from the group consisting of a sodium alginate fragment, a xanthan gum fragment, a pectin fragment, a gellan gum fragment, a gum karaya fragment, a gum tragacanth fragment, a sodium carboxymethylcellulose fragment and any combination thereof.

In the process of synthesizing a synthetic polysaccharide, it would be appreciated that the degree of polymerisation and hence the polymer chain length of the synthetic polysaccharide may be, at least to some degree, pre-determined or controlled. Accordingly, such synthetic polysaccharides may be tailored to the exact functional requirements desirable for the food grade thickener of the present invention (e.g., their ability to suitably inhibit the viscosity imparted by a thickening agent).

The polysaccharide fragment described herein may be produced by any method known in the art. By way of example, naturally occurring polysaccharides of high average molecular weight (e.g., greater than 500,000) and hence high viscosity can be reduced to a lower average molecular weight fragment thereof (e.g., less than 500 000) and hence lower viscosity by way of hydrolysis (e.g. acid hydrolysis). Any food grade acid, such as citric acid, hydrochloric acid, malic acid, tartaric acid, acetic acid, lactic acid, may be used in concentrations sufficient to achieve a pH below 4, preferably between 1.0 and 3.0, and heated to between about 60 to about 120° C., preferably between about 80 to about 100° C., for approximately 5 to 6 hours, preferably 2 to 3 hours, until the desired level of hydrolysis is reached. Hyperbaric pressures, such as those above 1 atmosphere, can be used to accelerate the rate of hydrolysis and hence reduce overall processing times.

Alternatively, a polysaccharide fragment described herein may be produced by enzymatic digestion of a larger starting polysaccharide. Naturally occurring polysaccharides contain sugars in their fundamental structure (e.g., glucose, fructose, mannose, arabinose, galactose, rhamnose, glucuronic acid, galacturonic acid, xylose) joined together via various types of glycosidic bonds that are capable of being hydrolysed by various specific enzymes. It would be understood by the skilled artisan that the particular enzyme required for digestion of the starting polysaccharide typically depends on the specific sugar/sugar bond that is being targeted for hydrolysis and hence determines the extent of the reduction in molecular weight. Additionally, the optimum conditions to achieve the most effective degree of hydrolysis are also specific to the enzyme in question and the type of glycosidic bond it can hydrolyse.

The size of the polysaccharide fragment will typically be a function of various factors, such as the desire for a smaller polysaccharide that is more conveniently adapted to the inhibition of viscosity imparted by the thickening agent of the food grade thickener. In particular embodiments, the polysaccharide fragment comprises at least about 5, 10, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, or 2750 contiguous saccharide units, or any range therein, of the larger, starting polysaccharide.

Another method of classifying cellulosic polysaccharides is by the amount of viscosity they can produce for a given concentration. This can be explained with reference to sodium carboxymethyl cellulose (CMC) as follows, but is equally applicable to all cellulosic polysaccharides. Sodium carboxymethyl cellulose (CMC) can be categorised into the following groups depending on the viscosity that a solution of a certain concentration (typically, 2% w/w) produces:
High Viscosity CMC—50 000 to 8 000 mPa·s for a 2% w/w solution, DP>1000, average molecular weight>250 000
Medium Viscosity CMC—8 000 to 800 mPa·s for a 2% w/w solution, DP 750 -1000, average molecular weight 175 000-250 000
Low Viscosity CMC—100-800 mPa·s for a 2% w/w solution, DP 500-750, average molecular weight 175 000-125 000, and
Ultra-low Viscosity CMC—100-1 mPa·s for a 2% w/w solution, DP<500, average molecular weight<125 000.

There is a relationship between the viscosity of a 2% w/w solution of CMC and its degree of polymerisation (and hence its molecular weight), therefore, it is equally valid to specify the viscosity inhibition ability of a CMC (and in fact all cellulosic polysaccharides) by the effective range of a 2% solution of that CMC. Low viscosity CMC, medium viscosity CMC and high viscosity CMC produce 2% solution viscosities that are too high to be able to produce a flowable liquid thickener solution. Instead, they produce a thick and non-flowable paste that is hard to disperse into liquid food. FIG. 5 illustrates the typical viscosity versus concentration profiles for the four groups of CMC gums described above

The food grade thickener provided herein may comprise one or more polysaccharides, including synthetic polysaccharides, naturally occurring polysaccharides, polysaccharide fragments and/or ionic polysaccharides. By way of example, the food grade thickener may comprise 1, 2, 3, 4, 5 or more polysaccharides, such as those described herein.

Mixing the Second Polysaccharide into the Continuous Phase Thereby Forming a Gelled Mixture

In preferred embodiments, the second polysaccharide is mixed into the continuous phase under low shear conditions to solubilise the second polysaccharide and form the gelled mixture. A suitable mixing speed is 10 to 200 rpm. However, it will be appreciated that a variety of equipment may be used, and various mixing speeds may be employed. It is preferable if the shear conditions are minimised or kept low. This is a departure from the prior art, which uses high mixing speeds and/or high shear conditions using what is understood to be rotor stator at 2000 rpm. Using high mixing speeds and/or high shear conditions can express too much viscosity from the second polysaccharide, which increases the apparent viscosity above a target of about 4000 to 5000 cPs of the food grade thickener of the invention, and is an irreversible viscosity and/or rheology change to the system. As discussed further below, it is preferable for the food grade thickener of the invention to have an apparent viscosity around 4000 to 5000 cPs, which provides rapid dispersion in a target foodstuff.

As can be seen in FIG. 2, incorporating the polysaccharide under high shear conditions can reduce the clarity of the composition. FIG. 2a shows a CMC solution which was incorporated using low shear mixing (10-200 rpm), and subsequently hydrolysed for 0, 8, 10, 12 and 14 hours. The writing on the reverse of the label can clearly be seen through the solution, indicating a clarity that is almost that of water. In comparison, FIG. 2b shows a CMC solution which was incorporated using high shear mixing (7600-10200 rpm) and subsequently hydrolysed for 0, 12, 24, 36 and 48 hours. These images clearly show that the solutions of FIG. 2a have a substantially higher clarity than those of FIG. 2b. The writing on the reverse of the label can only be seen through the solution (T-0), indicating a clarity that is almost that of water, but the solutions are completely or substantially opaque for the other experiments.

It will be appreciated that the method of dissolving the second polysaccharide and the extent of agitation (shear) during dissolution, may influence the final viscosity of the food grade thickener of the invention. Additionally, the solvent, the chemical composition of the second polysaccharide and/or the shear history of the final solution may influence the dissolution properties of the second polysaccharide.

### Hydrolysis Stage

In a further step of a preferred form of the method of the invention, the gelled mixture is hydrolysed to reduce the viscosity of the gelled mixture. The skilled person will appreciate that a variety of methods may be used to hydrolyse the gelled mixture. However, in one preferred embodiment, the temperature of the gelled mixture is preferably increased to about 95° C., or to around 90° C., or to exactly 90° C., or to 89, 88, 87, 86, 85, 84, 83, 82, 81, 80, 75, 70, 65, 60, 55 or 50° C. In some preferred embodiments, the time at which the gelled mixture is held at temperature is 2, 3, 4, 5, 6, 7, 8, 9, 10, 11, 12, 13, 14, 15, 16, 17, 18, 19, 20, 21, 22, 23, 24, 36, 48, 60 or 72 hours, or any time in between.

In preferred embodiments, the hydrolysis stage is conducted for a sufficient time and at a sufficient temperature so that the viscosity is reduced to a predetermined viscosity, which is preferably in the range of about 80-90 cPs (measured that at 20° C., 10 rpm with spindle #1, Brookfield rotational viscometer). In other preferred embodiments, the viscosity is brought into a range of 40-50, 50-60, 60-70, 70-80, 80-90, 90-100, 100-110, 110-120, 120-130, 130-140, or 140-150 cP. Typically, the viscosity of the gelled mixture is around 250-300 cPs (measured that at 20° C., 20 rpm with spindle #3, Brookfield rotational viscometer), meaning the hydrolysis stage provides a viscosity reduction of around 50%, but may be conducted to provide a reduction of 20, 30, 40, 50, 60, 70, 80 or 90% in viscosity. It will be appreciated that the gelled mixture may have a higher or lower viscosity than around 250-300 cPs, and irrespective of the starting viscosity of the gelled mixture the hydrolysis stage is conducted so as to preferably achieve a target viscosity of around 60-120 cPs.

In one preferred embodiment of the invention there is the proviso that that the hydrolysed material is not precipitated and recovered, and that recovered material then used to create a continuous phase into which a gum is added.

It will be appreciated that this is a convenient point in the method of the invention to provide other food grade additives to the hydrolysed gelled mixture, for example to adjust pH and add preservatives.

Another potential method for hydrolysing the second polysaccharide is by enzymatic hydrolysis. Many enzymes exist that can cleave specific bonds in polysaccharide molecules. When cleaved by the action of these enzymes, the polysaccharide effectively decreases its molecular weight becoming a smaller molecule that is potentially less effective at binding water and restricting its movement, that is, reducing the apparent viscosity that the polysaccharide can express. Table 1 below shows the enzymes that are capable of cleaving various food polysaccharides.

TABLE 1
Polysaccharides with the enzymes that are capable of hydrolysing them:

| | Enzyme(s) that are Capable of Cleaving |
|---|---|
| Polysaccharide | the polysaccharide |
| Xanthan gum | Xanthan lyase (4,5-transeliminase) and |
| | xanthan depolymerase (endo-1,4-□-D- |
| | glucanase) |
| Agar | Glycoside hydrolases |
| Carrageenan | Kappa-carrageenase, lamda-carrageenase, |
| | and iota-carrageenase |
| Gellan gum | Gellanases |
| Galactomannans (fenugreek gum, guar gum, | β-mannanase, β-mannosidase, and α- |
| tara gum and locust bean gum) | galactosidase |
| Alginates | Alginate lyases |
| Pectins | Pectinases |
| Cellulosics (microcrystalline cellulose, sodium | Cellulase enzymes |
| carboxymethylcellulose, methyl cellulose, | |
| hydroxypropylmethylcellulose, | |
| hydroxyproylcellulose, | |
| methylethylcellulose) | |
| Furcellaran | Kappa-carrageenase |
| Gum ghatti | Beta-D-galactopyrannases |
| Tragacanth gum | Pectinases |

The optimal conditions for enzyme hydrolysis are different for each enzyme but are well known to the person skilled in the art. The person skilled in the art can select the optimal conditions for enzyme hydrolysis and monitor the progress of the hydrolysis reaction by monitoring the reduction in viscosity of the polysaccharide solution. Generally, at least a 2-fold reduction in viscosity will be observed, typically at least a 3-fold reduction and potentially reductions in viscosity as much as 5-fold or 7-fold.

### Gum Addition

Preferably the conditions of the hydrolysed gelled mixture are such that the gum only partially expresses its viscosity. In preferred embodiments, around 4 to 8 wt. % of gum is added. It will be appreciated that other concentrations may be used, such as 2, 3, 4, 5, 6, 7, 8, 9, 10, 11, 12, 13, 14, 15, 16, 17, 18, 19, 20, 21, 22, 23, 24, 25, 26, 27, 28, 29 or wt. %.

Preferably the gum is added to the hydrolysed gelled mixture under low shear conditions and incorporated to partially hydrate the gum. A suitable mixing speed is 10-200 rpm. However, will be appreciated that a variety of mixing speeds may be employed, which may comprise low shear conditions, to achieve a target final viscosity of around 4000 to 5000 cPs.

Without wishing to be bound by theory, the method of the invention seems to provide optimal tertiary and quaternary structures of the polysaccharides that limit the degree of gum hydration, thereby providing a resistance or tolerance to shear during pumping and yet simultaneously allows facilitate rapid dispersion in the target foodstuff, and such that the peak viscosity of the gum is reached under gentle agitation conditions, e.g., 30 seconds at 100-150 BPM.

### Gums and Thickening Agents

According to the method of the invention, a gum is added to the hydrolysed gelled mixture. It will be appreciated that the terms “gum” and “thickening agent” are used interchangeably herein. In one embodiment a single gum is added to the hydrolysed gelled mixture. However, it will be appreciated that one or more gums may be added. The gum may be selected from the group consisting of agar, alginic acid, carrageenan, guar gum, gum tragacanth, gum ghatti, microcrystalline cellulose, sodium carboxymethylcellulose, methyl cellulose, hydroxypropylmethylcellulose, hydroxyproylcellulose, methylethylcellulose, gum karaya, locust bean gum, tara gum, psyllium seed gum, quince seed gum, a pectin, furcellaran, gellan gum, konjac, sodium alginate, galactomannans (fenugreek gum, guar gum, tara gum and locust bean gum), xanthan gum and any combination thereof.

In some embodiments the gum is selected from the group consisting of alginic acid, xanthan gum, sodium carboxymethyl cellulose, a pectin, gellan gum, gum karaya, gum tragacanth and any combination thereof.

An aspect of the invention is that the gum is included in an amount such that the food grade thickener has a lower viscosity than that of the food grade thickener comprising the gum only.

Preferably the gelled continuous phase resulting from the combined action of the first and second polysaccharides that have been hydrolysed enables an addition of a gum such that the viscosity of the food grade thickener is at least 50%, 40%, 30%, 20% 10% or 5% of that of a composition comprising the gum only.

The ability of a thickening agent, such as those described herein, to increase the viscosity of an aqueous solution is typically determined, at least in part, by its ability to bind water molecules and/or to unfurl from a rigid crystal-like structure into a network of entangled random polymer chains that can form strong and weak associations that impede the movement of water. The impediment of water molecules to move freely in solution manifests itself as an increase in viscosity. To this end, the present invention is predicated, at least in part, on the finding that the water binding ability of particular thickening agents and/or the degree to which these particular thickening agents can unfurl from a rigid crystal-like structure into a network of entangled random polymer chains can be modulated and/or controlled to produce specific degrees of viscosity inhibition by the addition of one or a plurality of polysaccharides, such as those described herein. Additionally, these polysaccharides may further control the rate and extent that their viscosity inhibition is released and/or reversed upon dilution of the food grade thickener.

In particular embodiments of the aforementioned aspects, the food grade thickener comprises one or more gums. For example, the food grade thickener may comprise 1, 2, 3, 4, 5 or more thickening agents.

### Food Grade Thickener

The food grade thickener of the invention has one of more of the following properties:
a) Shelf stable: >6-month shelf life in a hermetically sealed container.
b) Flowable: The apparent viscosity of the concentrate is such that it may be poured.
c) Dispersible: The food grade thickener can be incorporated into a target aqueous foodstuff with gentle stirring (e.g., 100 BPM in <1 min) to express viscosity within 30 seconds.
d) Pumpable: The food grade thickener can be pumped through a dispensing pump (which inherently imparts shear) without negatively impacting the dispersibility. The food grade thickener of the invention resists shear on pumping.
e) Clarity: The food grade thickener of the invention is clear and transparent. For example, a 7% solution in water has a >98% transmittance at 650 nm (1 cm path length). The prior art is silent on clarity, and as shown below does not have the clarity achieved by the food grade thickener of the invention.
f) Homogenous: The food grade thickener is substantially homogenous, in that it is substantially consistent with no lumps or highly gelled domains or aggregates within the bulk.

Preferably, the food grade thickener referred to herein is stable for at least six months and up to at least two years at room temperature. Because the thickener composition of the invention is stable, without significant degradation in the performance of the thickening agent, the viscosity remains constant for a commercially reasonable period of time. Accordingly, the formulation can be provided as a packaged product per se, such as in a metered pump dispenser, to the end user. To this end, the end user can reliably calculate the amount of the good grade thickener of the invention to add to a food or beverage to achieve a desired end viscosity thereof. The food grade thickener of the invention is then easily dispensed and easily mixed into the foodstuff to give the desired end product. The ability to package and use the food grade thickener in this way is a result of the combined presence of the thickening agent and polysaccharide which inhibits the expression of the viscosity of the thickening agent and provides distinct benefits in use over traditional sachets of powdered or gel-like thickener which are notoriously difficult to measure out accurately, when the exact pack size is not appropriate, and to incorporate into liquid foodstuffs.

In some embodiments, the food grade thickener has a viscosity of about 100, 200, 300,

400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9500, 10000, 11000, 12000, 13000, 14000, 15000, 16000, 17000, 18000, 19000, 20000 cP or any range therein. Preferably, the food grade thickener has a viscosity of between about 500 cP to about 10 000 cP. More preferably, the food grade thickener has a viscosity of between about 2000 cP to about 8000 cP. In one preferred embodiment, the food grade thickener is of a viscosity that is pump dispensable.

Stability of the liquid food grade thickener of the invention over time may be indicated by the retention of colour (if any), flavour (if any), separation (if any), microbiological spoilage (if any), viscosity and/or clarity of the food grade thickener. Additionally, or alternatively, stability of the food grade thickener may be determined by the ability of the composition to impart viscosity to a predetermined level when added to a foodstuff. The stability of the food grade thickener can be determined by using any of the techniques available to a person skilled in art of food science, including microbiological testing to measure the extent and rate of microbiological spoilage; visual inspection for physical changes such as separation and/or sedimentation; sensory evaluation to determine colour, flavour and/or clarity changes; and viscosity measurement using a Bostwick Consistometer or Brookfield Viscometer or similar device.

According to a second aspect, the present invention provides a food grade thickener when produced by the method according to the first aspect.

According to a third aspect, the present invention provides a method for increasing the viscosity of an aqueous liquid or aqueous liquid solid mixture foodstuff, the method comprising the step of adding to the foodstuff the food grade thickener produced by the method according to the first aspect.

According to a fourth aspect, the present invention provides a method of treating a subject suffering from a mastication and/or deglutition disease, disorder or condition, the method comprising the step of administering a foodstuff to the subject, wherein the foodstuff comprises the food grade thickener produced by the method according to the first aspect.

According to a fifth aspect, the present invention provides the use of the food grade thickener produced by the method according to the first aspect in the manufacture of a medicament for the treatment or amelioration of a mastication and/or deglutition disease, disorder or condition.

According to a sixth aspect, the present invention provides a method of overcoming or ameliorating difficulties in swallowing in a patient in need of such treatment, comprising the step of thickening a food or beverage with the food grade thickener produced by the method according to the first aspect for consumption by said patient.

According to a seventh aspect, the present invention provides the use of the food grade thickener produced by the method according to the first aspect in the manufacture of a medicament for overcoming or ameliorating difficulties in swallowing in a patient in need of such treatment.

According to an eighth aspect, the present invention provides a storage and delivery system for a food grade thickener, comprising:
a.) a container containing the food grade thickener produced by the method according to the first aspect, and
b.) a pump dispenser sealingly attached to the container, said dispenser comprising a valve for inhibiting or preventing drying of the composition in the container.

According to a ninth aspect, the present invention provides a kit or a storage and delivery system for a food grade thickener, comprising:
a.) a container containing the food grade thickener produced by the method according to the first aspect, and
b.) a pump dispenser for attachment to the container,
wherein said pump dispenser comprises a valve for inhibiting or preventing drying of the composition in the container.

According to a tenth aspect, the present invention provides a method of delivering a food grade thickener to an aqueous liquid or aqueous liquid solid mixture foodstuff, the method comprising the steps of:
a.) providing a container containing the food grade thickener produced by the method according to the first aspect, and
b.) applying a force to the pump dispenser to thereby deliver one or more doses of a predetermined volume of the food grade thickener to the foodstuff.

According to an eleventh aspect, the present invention provides a swallowing disorder assisting or ameliorating composition, comprising a pourable, food grade thickener, having an apparent viscosity of about less than about 5,000 cPs measured at 20° C. using a #3 spindle at 5 rpm, and a resistance to flow of greater than about 12 cm at 20° C. at 30 seconds measured using a Bostwick consistometer and having a transmittance of >90% at 650 nm when measured using a 1 cm path length.

According to a twelfth aspect, the present invention provides a method for providing a food grade thickener, the method comprising the steps of:
establishing an aqueous continuous phase of a first polysaccharide,
adding a second polysaccharide to the continuous phase thereby forming a gelled mixture,
hydrolysing the gelled mixture to reduce the viscosity of the gelled mixture, and

adding a gum to the hydrolysed gelled mixture under conditions such that the gum only partially expresses its viscosity, thereby forming the food grade thickener.

### Treatment of Swallowing Disorders

Trouble with swallowing generally refers to two problems:
a.) dysphagia—the sensation of food or fluid being regurgitated or stuck in the chest, or any throat discoordination leading to coughing or choking during swallowing, and
b.) odynophagia—pain in throat or chest during swallowing.

Swallowing disorders may result from a lack of coordination of the nerves or muscles, or sometimes from infections and tumours. Symptoms of swallowing disorders include:
Dysphagia—a sense of food “sticking” on the way down and difficulty passing food or liquid from the mouth to the esophagus to the stomach,

Coughing during or immediately after swallowing,

Choking—a feeling of food or liquid sticking in the throat or oesophagus followed by coughing,
Regurgitation—the return of food or liquid back to the mouth or pharynx after it successfully passed.
Nasal regurgitation—when food or fluid comes up into the nose; this occurs when the nasopharynx does not close properly

Other symptoms may include: sore throat, hoarseness, shortness of breath and chest discomfort or pain.

The food grade thickener of the present invention provides an aid to a patient suffering from such swallowing disorders by assisting with ingestion of a foodstuff. In particular, the treatment comprises the step of modifying the foodstuff so as to avoid or substantially avoid one of more of the symptoms described above. Preferably the target foodstuff having increased viscosity as a consequence of use of the inventive food grade thickener disclosed herein is for feeding a subject suffering from a mastication and/or deglutition disease, disorder or condition. Preferably, the mastication and/or deglutition disease, disorder or condition is or comprises dysphagia.

Preferably, the food grade thickener, according to the present invention, is added to an aqueous liquid or aqueous liquid solid mixture foodstuff for feeding to a subject suffering from a mastication and/or deglutition disease, disorder or condition. Preferably, the mastication and/or deglutition disease, disorder or condition is or comprises dysphagia. In some embodiments, the food grade thickener is separated into appropriate individual portions, such as sachets, or is pump dispensable.

It would be readily understood that dysphagia is a condition where the process of swallowing is impaired. During eating, this can lead to the entry of liquid or solid food into the trachea and subsequently the lungs of the sufferer potentially leading to aspiration pneumonia. Dysphagia can occur at any age, but is most common in the elderly, especially if they have suffered a stroke or have dementia. One management strategy for suffers of dysphagia is to consume foods that are texture modified (i.e., thickened foods and beverages) that slow the swallowing reflex and allow the windpipe time to close before the food passes, thereby preventing aspiration.

### Storage and Delivery System for a Food Grade Thickener

A further aspect of the invention is the provision of the food grade thickener of the invention in a container, and wherein the container comprises a pump dispenser sealingly attached to the container to thereby provide a substantially hermetically sealed system. The dispenser preferably comprises a valve for inhibiting or preventing drying of the composition in the container.

Preferably the dispenser comprises a dispenser tip, the dispenser tip including the valve disposed therein. Suitably, the aforementioned valve is or comprises a self-sealing valve. In particular embodiments, the aforementioned valve is selected from the group consisting of a cross-slit valve, a ball valve, a flapper valve, an umbrella valve, a duck bill valve, a reed valve and any combination thereof. In particular embodiments of the invention, the valve is biased to a closed position and is actuated to an open position upon application of a force to the pump dispenser forcing said composition to flow through the valve.

The storage and delivery system of the invention preferably comprises a pump dispenser, or another sealed delivery system as are known in the art, that: (1) delivers a consistent dose or volume (e.g., +/−3% to 5% by weight) of the food grade thickener described herein upon use and through the entire content or volume of the storage and delivery system in which it is sold; and (2) is able to protect the food grade thickener from the drying effect of an atmosphere or environment having a relative humidity below 95% whilst contained or stored within the storage and delivery system.

An advantage of storing the food grade thickener of the invention in a hermetically sealed container is an improvement to long-term stability. For example, the food grade thickener of the invention is stable for at least 6 months at room temperature.

In another embodiment of the invention, the food grade thickener of the invention is stored and/or delivered by a sachet or the like. In one embodiment, the sachet includes a dispenser in the form of a tear-away pour spout.

In preferred embodiments of the invention the food grade thickener maintains a water activity of greater than 95%. It would be readily understood, that water activity or aw is defined as the ratio of the partial vapour pressure of water in a material to the standard state partial vapour pressure of water at the same temperature. Additionally, water generally migrates from areas of high water activity to areas of low water activity until an equilibrium is reached. For example, the food grade thickener provided herein has a water activity in excess of 95% (e.g., about or in excess of 95.5%, 96%, 96.5%, 97%, 97.5%, 98%, 98.5%, 99%, 99.5% and any range therein), which then typically requires protection from atmospheres or environments with a relative humidity of less than 95%, so as to prevent the food grade thickener from drying out during storage and before delivery or dispensing.

Accordingly, the storage and delivery system preferably provides a relatively precise and/or accurate dose or volume of the food grade thickener to the desired foodstuff. In particular embodiments, delivery of the food grade thickener by the storage and delivery system of the invention to the foodstuff results in a viscosity thereof that is within at least +/'7.5% (e.g., 5 +/−0.5%, 1%, 1%, 1 0.5%, 2%, 2.5%, 3%, 3.5%, 4%, 4.5%, 5%, 5.5%, 6%, 6.5%, 7%, 7.5% and any range therein) of the desired or pre-determined viscosity of said foodstuff. More preferably, delivery of the food grade thickener by the storage and delivery system of the invention to the foodstuff results in a viscosity thereof that is within at least +/−3.5% of the desired or pre-determined viscosity of said foodstuff.

In some embodiments, the storage and delivery system may deliver a volume of 4.5, 5, 6, 6.5, 7, 7.5, 8, 8.5, 9, 9.5, 10, 11, 12, 13, 14, 15 ,16, 17, 18, 19, 20, 21, 22, 23, 24, 25, 26, 27, 28, 29, 30, 31, 32, 33, 34, 35, 36, 37, 38, 39 or 40 mL, or any amount in between of the food grade thickener to the target foodstuff.

In some embodiments, the storage and delivery system will deliver shear at a rate of 50, 100, 200, 300, 400, 500, 600, 700, 800, 900 or 1000 s⁻¹, or any shear rate in between. It will be appreciated that the degree of shear experienced by the food grade thickener is related to the inner working of the pumping apparatus, and will vary between different pumping apparatus.

### Use of the Food Grade Thickener to Increase the Viscosity of a Foodstuff

In some embodiments, a sufficient amount of food grade thickener is added to the target foodstuff to achieve a desired target viscosity, for example, 1 to 30 wt %. In some embodiments, the amount of food grade thickener that is added to the foodstuff is in the range of 1 to 5, 6 to 10, 11 to 15, 16 to 20, 21 to 25 or 26 to 30 wt % (based on the total mass of the solution) or any range in between.

In some embodiments, the viscosity of said foodstuff, upon addition of the food grade thickener of the invention, is increased to at least 95, 100, 110, 120, 130, 140, 150, 175, 200, 250, 300, 350,400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3100, 3200, 3300, 3400 or 3500 cP, or any range therein.

### BRIEF DESCRIPTION OF THE DRAWINGS

Preferred embodiments of the invention will now be described, by way of example only, with reference to the accompanying drawings in which:

FIG. 1 shows a flowchart of the method of the claimed invention, indicating the steps of: continuous phase formation, gel formation, hydrolysis and gum addition.

FIG. 2a shows various CMC solutions (pH 3.6) solubilised with minimal shear (10-200 rpm) for 1 hour and subsequently hydrolysed at 90° C. for (L-R) Time 0 (>10 mL), 8 hours (5.8 mL), 10 hours (5.6 mL), 12 hours (5.0 mL) and 14 hours (4.2 mL). The values in brackets are the respective volume remaining in flow test performed according to IDDSI flow test (10 seconds of flow) in mL. FIG. 2b shows various CMC solutions (pH 3.6) solubilised with high shear (7600-10200 rpm) for 1 hour and subsequently hydrolysed at 90° C. for (L-R) Time=0 hours (>10 mL), 12 hours (3.4 mL), 24 hours (0.8 mL), 36 hours (0.3 mL) and 48 hours (0 mL). The values in brackets are the respective volume remaining in flow test performed according to IDDSI flow test (10 seconds of flow) in mL.

FIG. 3 shows the effect of various compositions of the prior art and the invention when mixed in a 1:5 ratio with water, showing a) the claimed invention, and b) h) the comparative example compositions produced according to the teachings of JP2007 using food grade polysaccharides.

FIG. 4 shows the stability comparative examples 1-8 over a period of 24 hours, showing that none of the compositions of comparative examples 1-8 maintain stability over this period.

FIG. 5 is a histogram diagram which illustrates the typical viscosity versus concentration profiles for the four groups of sodium carboxymethylcellulose (CMC) gums;

FIG. 6 is a graph showing the viscosity of a 5% xanthan gum solution as a function of acid hydrolysed sodium carboxymethylcellulose concentration;

FIG. 7 is a graph showing the viscosity of a 5% xanthan gum solution as a function of acid hydrolysed sodium alginate concentration;

FIG. 8 is a graph showing the viscosity of a 5% xanthan gum solution as a function of enzyme hydrolysed xanthan gum concentration;

FIG. 9 is a graph showing the viscosity of a 5% xanthan gum solution as a function of enzyme hydrolysed guar gum concentration;

FIG. 10 is a graph showing the viscosity of a 5% xanthan gum solution as a function of methyl ethyl cellulose (DP=250) concentration;

FIG. 11 is a graph showing the viscosity of a 5% xanthan gum solution as a function of sodium carboxymethylcellulose (DP-120-150) concentration;

FIG. 12 is a graph showing the viscosity of a 5% xanthan gum solution as a function of the concentration of the following mixture of low viscosity, highly soluble, polysaccharides—20 parts enzyme hydrolysed xanthan gum; 20 parts sodium carboxymethyl cellulose (DP=120−150); 10 parts acid hydrolysed sodium carboxymethyl cellulose; 20 parts acid hydrolysed sodium alginate; and 20 parts acid hydrolysed pectin; and

FIG. 13 is a graph showing the viscosity of an 8% sodium alginate solution as a

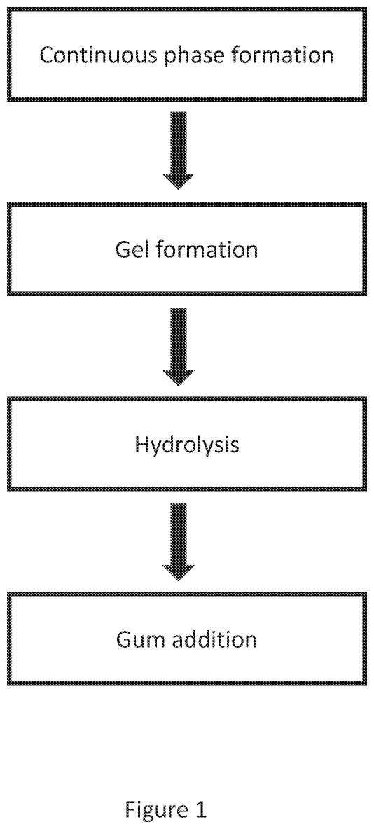

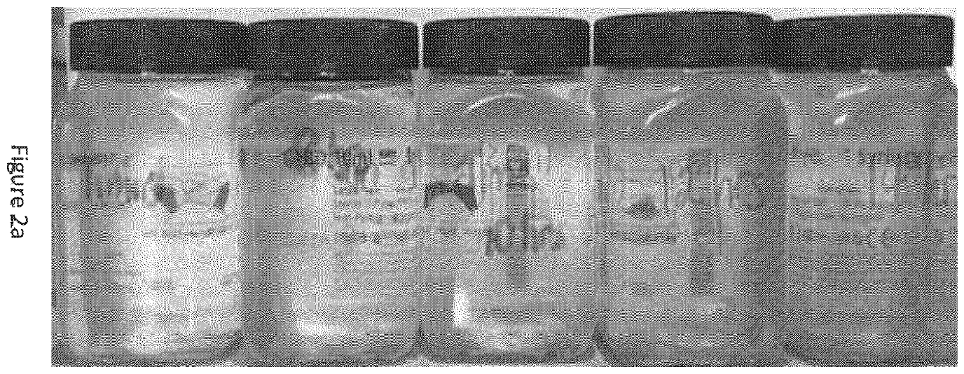

function of the concentration of the following mixture of low viscosity, highly soluble, polysaccharides—8.3 parts acid hydrolysed pectin; 8.3 parts hydroxypropyl methylcellulose (DP=200); and 8.3 parts enzyme hydrolysed guar gum.

### DEFINITIONS

In describing and claiming the present invention, the following terminology will be used in accordance with the definitions set out below. It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Unless the context clearly requires otherwise, throughout the description and the claims, the terms “comprise”, “comprising”, and the like are to be construed in an inclusive sense as opposed to an exclusive or exhaustive sense; that is to say, in the sense of “including, but not limited to”. For example, a composition, mixture, method or method that comprises a list of elements is not necessarily limited to only those elements but may include other elements not expressly listed or inherent to such composition, mixture, method or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition, method or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising”, it should be readily understood that (unless otherwise stated) the description should be interpreted to also describe such an invention using the terms “consisting essentially of” or “consisting of.” In other words, with respect to the terms “comprising”, “consisting of”, and “consisting essentially of”, where one of these three terms is used herein, the presently disclosed and claimed subject matter may include the use of either of the other two terms. Thus, in some embodiments not otherwise explicitly recited, any instance of “comprising” may be replaced by “consisting of” or, alternatively, by “consisting essentially of”.

Further, unless expressly stated to the contrary, “or” refers to an inclusive or and not to

an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be non-restrictive regarding the number of instances (i.e., occurrences) of the element or component. Therefore “a” or “an” should be read to include one or at least one, and the singular word form of the element or component also includes the plural unless the number is obviously meant to be singular.

Other than in the operating examples, or where otherwise indicated, all numbers expressing quantities of ingredients or reaction conditions used herein are to be understood as modified in all instances by the term “about”. The examples are not intended to limit the scope of the invention. In what follows, or where otherwise indicated, “%” will mean “weight %”, “ratio” will mean “weight ratio” and “parts” will mean “weight parts”.

The terms “predominantly” and “substantially” as used herein shall mean comprising more than 50% by weight, unless otherwise indicated.

As used herein, with reference to numbers in a range of numerals, the terms “about,” “approximately” and “substantially” are understood to refer to the range of −10% to +10% of the referenced number, preferably −5% to +5% of the referenced number, more preferably -1% to +1% of the referenced number, most preferably -0 .1% to +0 .1% of the referenced number. Moreover, with reference to numerical ranges, these terms should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, from 8 to 10, and so forth.

As used herein, wt. % refers to the weight of a particular component relative to total weight of the referenced composition.

The term “and/or” used in the context of “X and/or Y” should be interpreted as “X,” or “Y,” or “X and Y.” Similarly, “at least one of X or Y” should be interpreted as “X,” or “Y,” or “both X and Y.”

The terms “preferred” and “preferably” refer to embodiments of the invention that may

afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The complete disclosures of the patents, patent documents and publications cited herein are incorporated by reference in their entirety as if each were individually incorporated.

The term “polysaccharide”, as used herein, generally refers to polymers formed from about 10 to 500, 500 to 1000, 1000 to 2000, 2000 to 5000, 5000 to 10,000, 10,000 to 50,000, 50,000 to 100,000 or over 100,000 saccharide units linked to each other by hemiacetal or glycosidic bonds, or any range therein, for example, 10 to over 100,000 saccharide units. The polysaccharide may be either a straight chain, singly branched, or multiply branched wherein each branch may have additional secondary branches, and the monosaccharides may be standard D- or L-cyclic sugars in the pyranose (6-membered ring) or furanose (5-membered ring) forms such as D-fructose and D-galactose, respectively. Additionally, they may be cyclic sugar derivatives, deoxy sugars, sugar, sugar acids, or multi-derivatized sugars. As would be understood by the skilled artisan, polysaccharide preparations, and in particular those isolated from nature, typically comprise molecules that are heterogeneous in molecular weight.

The term “thickening agent” as used herein refers to any compound used to increase the viscosity of a liquid mixture and/or solution, and in particular, those for use in food applications, such as edible gums, vegetable gums and food grade polysaccharides.

As used herein, “synthetic polysaccharides” refer to chemically and/or enzymatically produced, derived and/or modified polysaccharides. In particular embodiments, the synthetic polysaccharide is selected from the group consisting of microcrystalline cellulose, sodium carboxymethylcellulose, methylcellulose, hydroxypropylmethylcellulose, hydroxypropylcellulose, methylethylcellulose, polyvinylpyrrolidone (PVP), a carboxyvinyl polymer, a methyl vinyl ether, a maleic anhydride polymer, an ethylene oxide polymer and any combination thereof.

A “naturally occurring polysaccharide”, as used herein, generally refers to a polysaccharide that is not modified from how it occurs in nature except for being isolated.

For the purposes of the present invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material includes material in native and recombinant form. In this regard, the naturally occurring polysaccharide may be synthesized so as to effectively replicate a polysaccharide as found in nature. The term “isolated” also encompasses terms such as “enriched” and “purified”.

By “polysaccharide fragment” is meant any complex carbohydrate which is formed, for example, by the enzymatic and/or chemical digestion of a larger, starting polysaccharide. Thus, while the fragment is always smaller than the starting polysaccharide from which it has been derived, no particular size limitation is implied on either the starting polysaccharide or the fragment thereof.

As used herein, the term “flowable” and like terms such as “flowability” refer to the ability of a substance to flow in a continuous steam without undue force under standard atmospheric conditions and temperatures.

As used herein, the term “pumpable” and like terms such as “pumpability” refer to the ability of a substance to flow under pressure through lines, nozzles, and passages of a pump apparatus and/or fittings thereof without irreversible deformation or adverse effects imparted on the material flowing through said lines, nozzles, and passages.

As used herein, the term “pourable” and like terms such as “pourability” refer to the ability of a substance to be poured or otherwise flow in a continuous steam without undue force under standard atmospheric conditions and temperatures.

As used herein, the term “dispersible” and like terms such as “dispersibility” refer to the ability of a substance to rapidly distribute evenly throughout a medium with minimal force without forming lumps or particulates.

As used herein, the units “cP”, “cPs”, “centipoise”, “mPa·s” and “millipascal-second” are understood to be interchangeable, and will be understood by the skilled person as describing the dynamic viscosity of a solution.

As used herein, the term “low shear” refers to conditions which use a relatively low speed (e.g., 2-300 rpm), preferably with a low shear agitator such as a hydrofoil impeller. It will be appreciated that a range of agitators would be appropriate, and the skilled person would be able to select one which imparts minimal shear to the solution. In some embodiments, the low shear is between 1 to 1000 rpm.

As used herein, the term “high shear” refers to conditions which use a relatively high mixing speed (e.g., >1500 rpm), and/or using a high shear agitator. In some embodiments, the high shear is >1,000 to 10000 rpm.

Unless stated otherwise, all viscosity and apparent viscosity measurements herein are performed at 20° C. at 5 rpm using a #3 spindle on a Brookfield rotational viscometer.

Unless stated otherwise, all resistance to flow measurements herein are performed at 20° C. over 30 seconds using a Bostwick consistometer.

Unless stated otherwise, all transmittance (clarity) measurements are performed on a Hach DR3900 spectrophotometer using 1 cm path length. However, it will appreciated that a range of spectrophotometers are appropriate, which would provide equivalent results.

As used herein, a Bostwick consistometer (“Bostwick”) is understood to relate to an instrument which determines the consistency of various materials by measuring the distance which a sample flows under its own weight. It will be appreciated that such an instrument complies with ASTM standards (ASTM F1080-93 (2019)).

As used herein, a Brookfield viscometer (“Brookfield”) is understood to relate to an instrument which determines the viscosity of a material by measuring the torque required to turn an object (such as a ‘spindle’),

### DETAILED DESCRIPTION

The skilled addressee will understand that the invention comprises the embodiments and features disclosed herein as well as all combinations and/or permutations of the disclosed embodiments and features.

The compositions produced by the method of the present invention may provide various advantages, such as one or more of the following, and are a significant advance over the prior art:
flowable,
pumpable,
homogenous (i.e., does not contain lumps or domains of undispersed gum and/or polysaccharide),
is dispersible into an aqueous liquid so that thickened liquid is homogenous,
has sufficient speed of hydration so the peak viscosity in the foodstuff is reached within a short time frame (ie around 30-60 sec) at low shear (i.e., 80-160 BPM with a fork/spoon),
has ability to withstand shear on delivery with a food grade pump,
does not separate over time,
is clear (no colour when dispersed in a feedstuff), and
imparts little or no odour or taste to the target foodstuff.

As discussed above, food grade thickeners are used typically in a patient's home, or in a hospital or aged care scenario. In this context, it is particularly convenient to provide a container with a pump, such that the patient or carer can simply dispense a predetermined quantity of thickener via the pump into a known amount of target foodstuff to thicken the foodstuff. It is important for the thickener to be flowable, and to have little or no odour or taste so as to negatively impact the foodstuff to which it is added. It is also highly preferable for the thickener to be clear and impart no colour to the target foodstuff, again so as to not negatively impact the foodstuff to which it is added. Furthermore, it is highly preferred if the thickener is easily dispersible into an aqueous liquid foodstuff so that thickened foodstuff is homogenous, and has sufficient speed of hydration so the peak viscosity is reached within a short time frame (i.e., around 30-60 seconds) at low shear (i.e., 80-160 BPM with a fork/spoon). When stored in a container/pump storage and delivery system, the thickener must be easily pumpable, and must withstand shear so as to not negatively impact the other desirable properties mentioned. Further still, it is highly desirable if the thickener is homogenous, i.e., does not contain lumps or domains of undispersed gum, either when in situ, or when delivered to the target foodstuff. Yet further still, it is highly desirable if the thickener is does not separate over time, either when in situ, or when delivered to the target foodstuff. Devising a food grade thickener that achieves these objectives is a significantly complex task, primarily because there are many competing objectives, and one needs to work within the limits of what the various ingredients inherently deliver to the composition. The applicant has devised a method that, in the preferred embodiments, delivers all of the above objectives.

In preferred embodiments, the food grade thickener described herein when added in a desirable amount to an aqueous liquid or aqueous liquid solid mixture foodstuff has a minimal or negligible impact on the particularly desirable attributes thereof, such as the original flavour and/or colour of the foodstuff, that may be attractive to the consumer. In this regard, the food grade thickener preferably makes little or no flavour and/or colour contribution to said foodstuff when added in a desirable amount thereto. Additionally, it is preferable that the amount of the food grade thickener to be added to a foodstuff to achieve a desirable viscosity thereof is relatively small so as to avoid diluting the flavour and/or colour characteristics of the foodstuff.

In preferred embodiments, the present invention provides a food grade thickener which is flowable. Advantageously, the food grade thickener is preferably of a viscosity such that it may be dispensed easily, such as from a pump, as well as being able to be dispersed with little or no agitation when added in a desired amount to an aqueous liquid or aqueous liquid solid mixture foodstuff. Preferably, the food grade thickener of the invention is concentrated and can accommodate a relatively high percentage of thickening agent without losing the flowable character of the composition. This further enables easy and accurate dispensing of the food grade thickener into the foodstuff of choice.

The following compositions exemplify the food grade thickener of the invention, and provide comparative examples to the prior art which show that the prior art cannot deliver on the objectives set out above, either individually, or collectively.

### EXAMPLES

The present invention will now be described with reference to the following examples which should be considered in all respects as illustrative and non-restrictive.

### Example 1: Method of the Invention

The inventive food grade thickener of the invention was prepared with the following method.
1. Adjust the pH of water to pH 3-4 with GDL.
2. Hydrolyse gellan gum in water (1:50-100 ratio).
3. Add hydrolysed gellan gum to acidified water. Add gelling cation (CaCl₂ 0.001%) and potassium sorbate (1000 ppm) preservative to solution.
4. Heat solution to 80° C.
5. Add around 2 to 8 wt. % of sodium CMC.
6. Mix for 1 hour under low shear 10-200 rpm to solubilise.
7. Increase temperature to around 90° C. for a sufficient time to hydrolyse the sodium CMC solution from a viscosity of around 250-300 cPs at 20° C. to around 80-90 cPs (measured at 10 rpm with spindle #1, on Brookfield rotational viscometer).
8. Adjust pH to 3.8-3.9 with GDL.
9. Reduce heat to 80° C. and incorporate 4-8% xanthan gum using low shear at 10-200 rpm, thereby producing the food grade thickener of the invention.

In a first step, the pH of water was adjusted to pH 3-4 with a common food grade acidifier (i.e., glucono delta-lactone; GDL). To the acidified water was added gellan gum, gelling salt and potassium sorbate (at 1000 ppm) to preserve mixture. The solution was then heated to 80° C. to activate the gellan gum.

Once the gellan gum was sufficiently combined into the solution the low molecular weight sodium CMCs were added. Around 2 to 8 wt. % of sodium CMC was added. Once combined, the solution was mixed under low shear conditions at 10-200 rpm to solubilise the CMCs.

Once solubilised, the viscosity of the solution was around 250-300 cPs, and the temperature was then increased to 90° C. and held for around 10 hours to hydrolyse sodium CMC solution to obtain a viscosity of around 80-90 cPs (measured at 20° C. at rpm with spindle #1, on Brookfield rotational viscometer).

In this stage, the temperature was increased to the target temperature of 90° C., ensuring that no hotspots were formed within the solution that exceeded the target temperature. At the conclusion of the heating stage, the pH was adjusted to 3.8-3.9 with GDL, and the temperature was reduced to 80° C., at which time 4-8% xanthan gum was added using low shear mixing at 10-200 rpm to incorporate, thereby producing the food grade thickener of the invention.

### Example 2: Composition 1

| Component | Amount (wt. %) |
|---|---|
| a CMC solution | 2 to 8 |
| Xanthan Gum | 3 to 12 |
| Gellan gum | 0.0325 |
| Potassium sorbate | 0.10 |
| Calcium chloride | 0.00325 |
| GDL solution (50% w/w) | 3.00 |
| Water | q.s. |

The inventive food grade thickener produced using the above method and composition 1 was a flowable liquid with an apparent viscosity of less than 5,000 cP. When lOg grams of the food grade thickener was added to 200 grams of water and stirred at 150 rpm for 30 seconds, the resulting solution reached a viscosity of about 80-100 mPa·s and had a transmittance of >90% measured at 650 nm with a 1 cm path length.

### Example 3: Composition 2

| Component | Amount (wt. %) |
|---|---|
| A CMC solution | 0.5 to 7 |
| Sodium alginate | 0.1 to 5 |
| Xanthan Gum | 6.5 |
| Gellan gum | 0.01 |
| Calcium chloride | 0.001 |
| water | q.s. |

The inventive food grade thickener produced using the above method and composition 2 was a flowable liquid with an apparent viscosity of about 14,600 cP (measured at 5 rpm with spindle #3, on Brookfield rotational viscometer). When 6 grams of the food grade thickener was added to 100 grams of water and stirred at 150 rpm for 30 seconds, the resulting solution reached a suitable viscosity and had a transmittance of >90% measured at 650 nm with a 1 cm path length.

### Example 4: Composition 3

| Component | Amount (wt. %) |
|---|---|
| Na CMC 30 cPs at a 2% solution | 3 |
| Na CMC 50 cPs at a 2% solution | 0.8 |
| Xanthan Gum | 5.7 |
| Potassium sorbate | 0.10 |
| GDL solution (50% w/w) | 3 |
| Water | 87.4 |

The inventive food grade thickener produced using the above method and composition 3 was a flowable liquid with an apparent viscosity of about 4000 cP. When 20 grams of the food grade thickener was added to 100 grams of water and stirred at 75 rpm for 30 seconds, the resulting solution reached an apparent viscosity of about 3000 cP and had a transmittance of about 84.7% measured at 650 nm with a 1 cm path length.

### Example 5: Composition 4

| Component | Amount (wt. %) |
|---|---|
| Na CMC | 3.5 |
| Xanthan Gum | 6.5 |
| Potassium sorbate | 0.10 |
| GDL solution (50% w/w) | 3.0 |
| Water | 86.9 |

The inventive food grade thickener produced using the above method and composition 4 was a flowable liquid with an apparent viscosity of about 8200 cP. When 5 grams of the food grade thickener was added to 100 grams of water and stirred at 150 rpm for 30 seconds, the resulting solution reached a suitable viscosity and had a transmittance of about 98% measured at 650 nm with a 1 cm path length.

### Example 6: Composition 5

| Component | Amount (wt. %) |
|---|---|
| Na CMC | 3.8 |
| Xanthan Gum | 7 |
| Gellan gum | 0.01 |
| Potassium sorbate | 0.10 |
| Calcium chloride | 0.001 |
| GDL solution (50% w/w) | 3.0 |
| Water | 86.089 |

The inventive food grade thickener produced using the above method and composition 5 was a flowable liquid with an apparent viscosity of about 14200 cP. When 5 grams of the food grade thickener was added to 100 grams of water and stirred at 150 rpm for 30 seconds, the resulting solution reached an appropriate viscosity and had a transmittance of about 99.1% measured at 650 nm with a 1 cm path length.

Comparative examples with JP2007

The following examples reproduce the method of JP2007 using commercially available food grade low molecular weight CMC's.

TABLE 2
Compositions of comparative examples

| | Concentration wt. % | | | | | | | |
|---|---|---|---|---|---|---|---|---|
| | Comparative | Comparative | Comparative | Comparative | Comparative | Comparative | Comparative | Comparative |
| Ingredient | example 1 | example 2 | example 3 | example 4 | example 5 | example 6 | example 7 | example 8 |
| Food grade | 5 | | 4 | 3 | | | 5 | 3 |
| CMC 1 | | | | | | | | |
| Food grade | | 5 | 1 | 0.8 | | 5 | | 0.8 |
| CMC 2 | | | | | | | | |
| Xanthan | 5 | 5 | 5 | 5.7 | 5 | 5 | 5 | 5.7 |
| gum | | | | | | | | |
| Potassium | | | | 0.1 | | | | 0.1 |
| sorbate | | | | | | | | |
| GDL | | | | 3.0 | | | | 3.0 |
| solution | | | | | | | | |
| Water | 90 | 90 | 90 | 87.4 | 95 | 90 | 90 | 87.4 |
| Total | 100 | 100 | 100 | 100 | 100 | 100 | 100 | 100 |

In Table 2, food grade CMC 1 has a viscosity of 30 cPs at a 2% solution and food grade CMC 2 has a viscosity of 50 cPs at a 2% solution.

Method for comparative examples 1-3 and 8.
1. Dissolve the food grade low molecular weight CMC(s) in water.
2. Stir with Silverson at 2000 rpm for 5 minutes as per JP2007.
3. Confirm no lumps.
4. Add xanthan gum and stir at 2000 rpm with Silverson for 1 minute.
5. Measure viscosity at 30 rpm (Brookfield rotational viscometer 20 rpm; Spindle 5 or 6 as viscosity dependent at 25° C.).

Method of comparative example 4.
1. Dissolve food grade low molecular weight CMC in water.
2. Stir with Silverson 2000 rpm 5 minutes.
3. Confirm no lumps.
4. Hydrolyse at 90° C. for 12 hours
5. Add xanthan gum and stir at 2000 rpm with Silverson for 1 minute.
6. Measure viscosity at 30 rpm (Brookfield rotational viscometer 20 rpm; Spindle 5 or 6 as viscosity dependent at 25° C.).

Method for comparative example 5
1. Add xanthan gum to water.
2. Stir at 2000 rpm with Silverson for 5 minutes.
3. Measure viscosity at 30 rpm (Brookfield rotational viscometer 30 rpm with spindle 5 or 6 as viscosity dependent at 25° C.).

Method for comparative example 6 & 7
1. Blend 2 food grade low molecular weight CMCs and xanthan gum.
2. Add mixture to water.
3. Stir with Silverson 2000 rpm for 1 minute.
4. Measure viscosity at 30 rpm (Brookfield rotational viscometer 30 rpm with spindle 5 or 6 as viscosity dependent at 25° C.).

A silverson mixer with a general purpose stator was used, which is considered to be equivalent to the ‘dispermix’ disclosed in JP2007 and, as no additional information was provided in the specification of JP2007, a generic stator attachment was utilised.

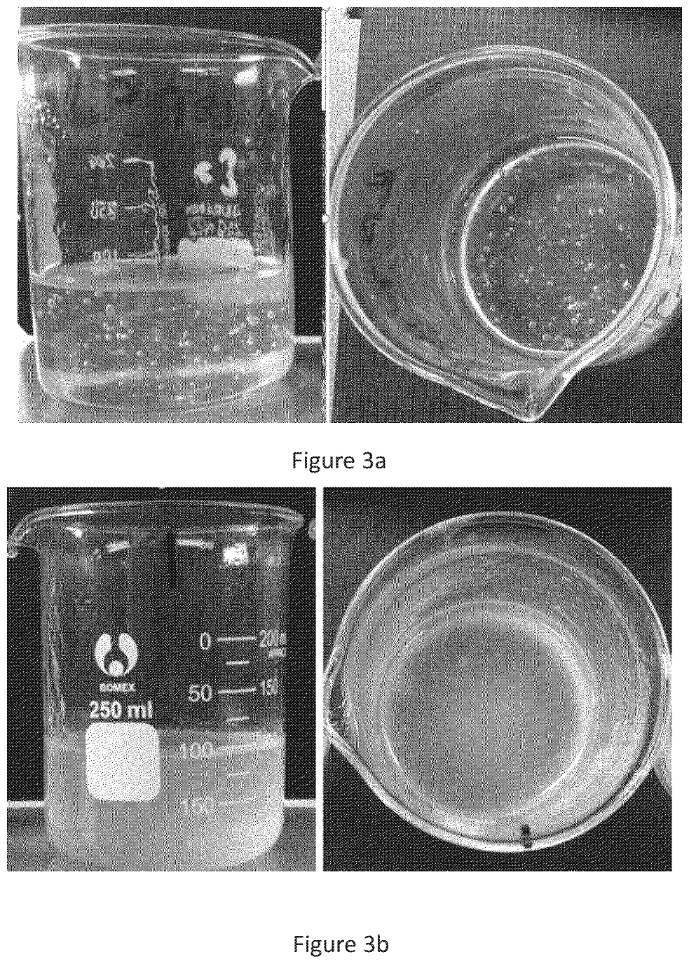

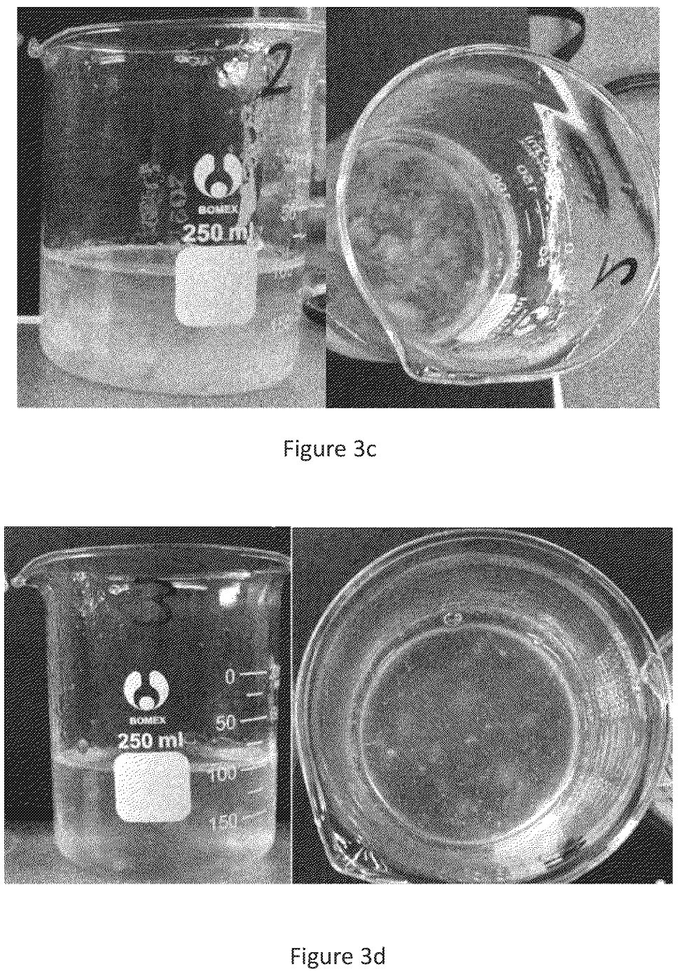

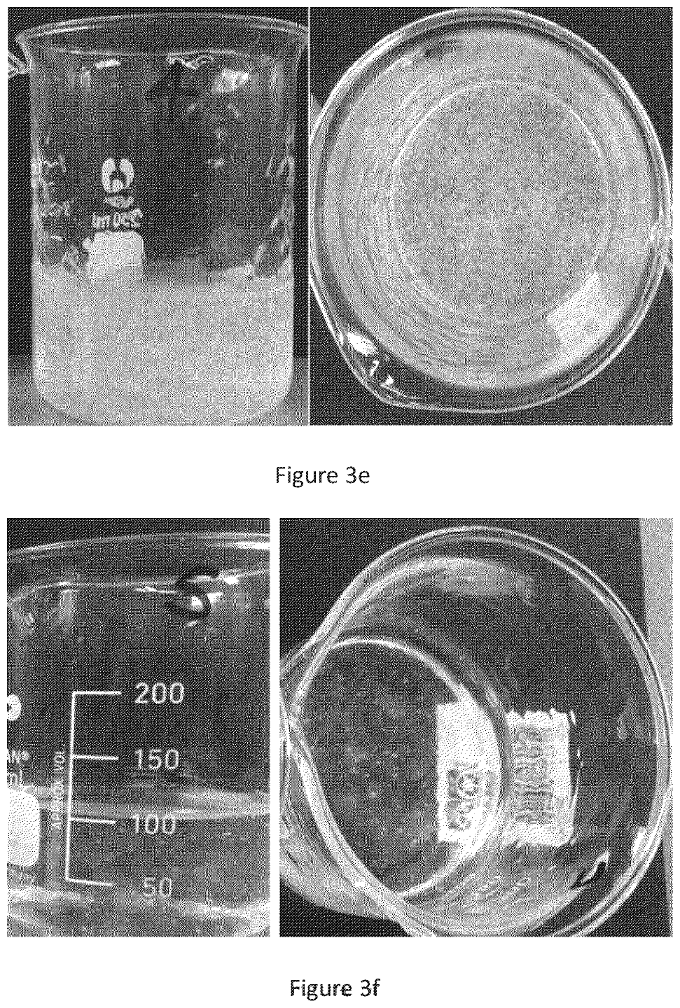

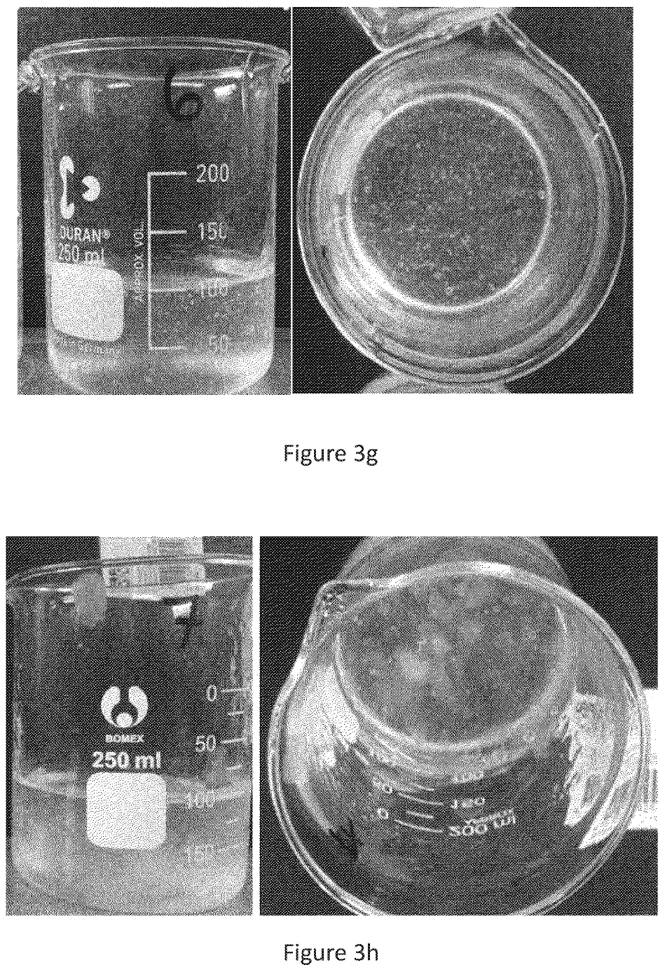

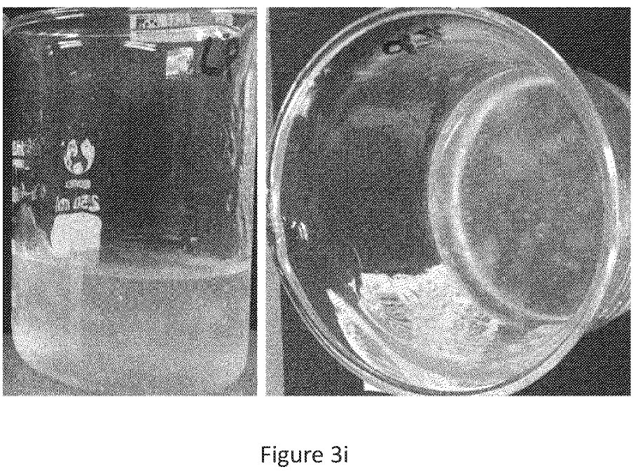

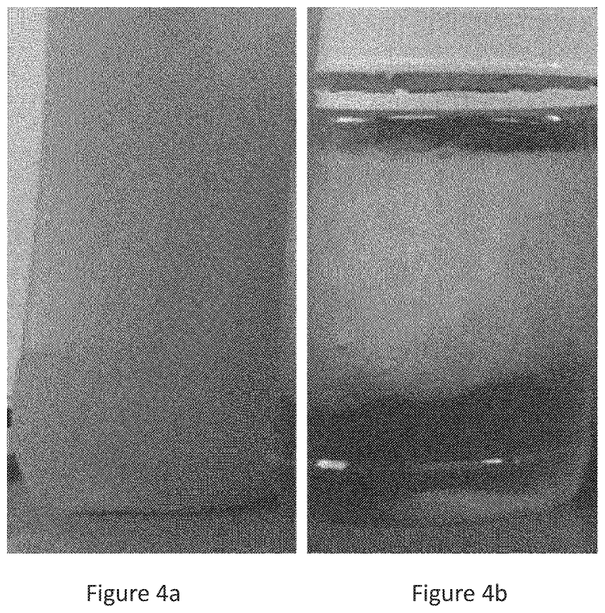

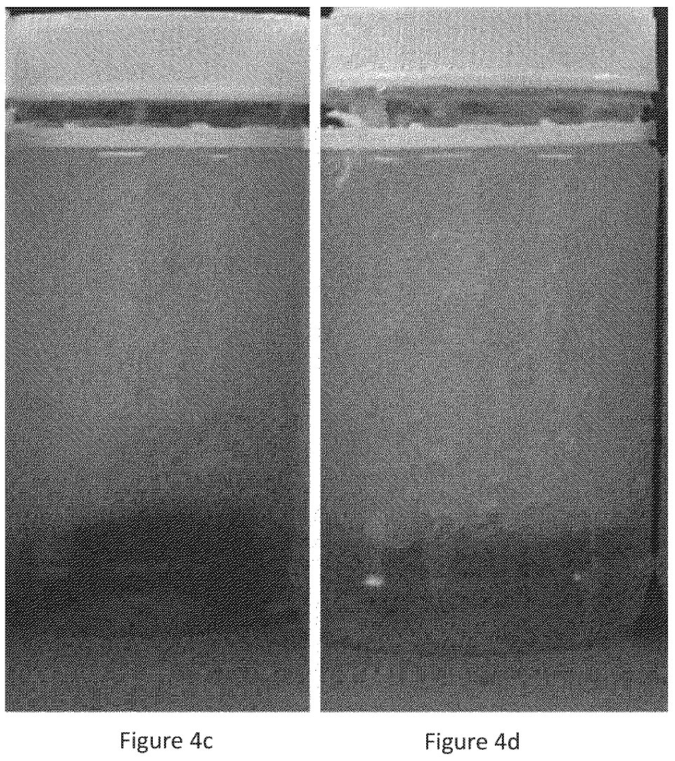

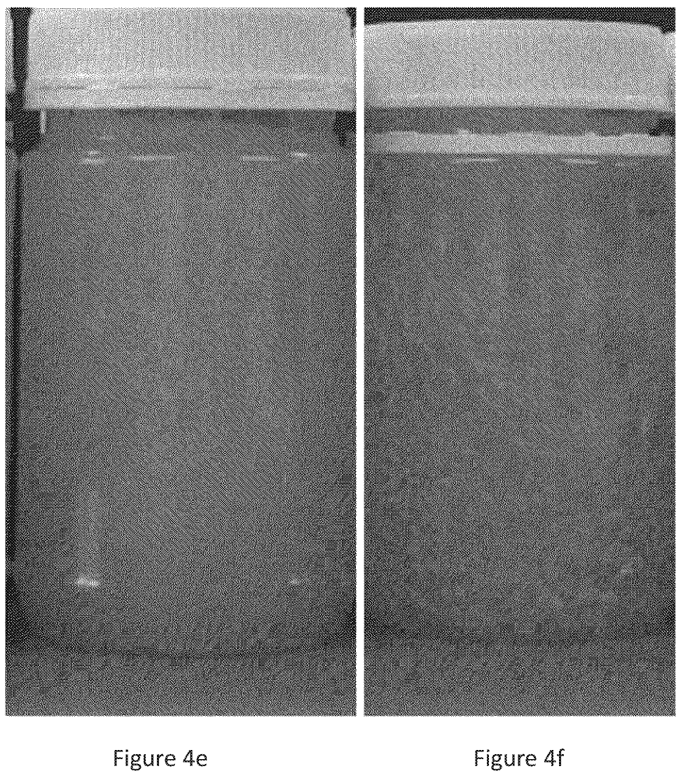

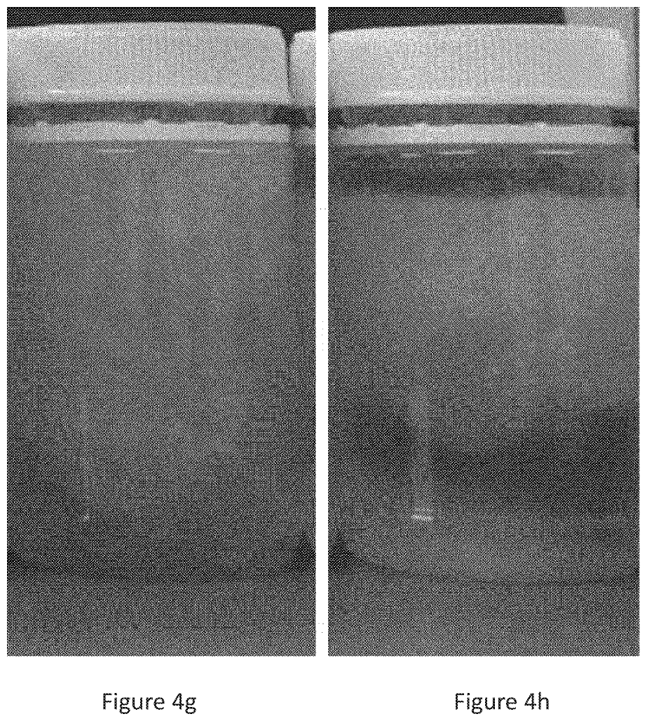

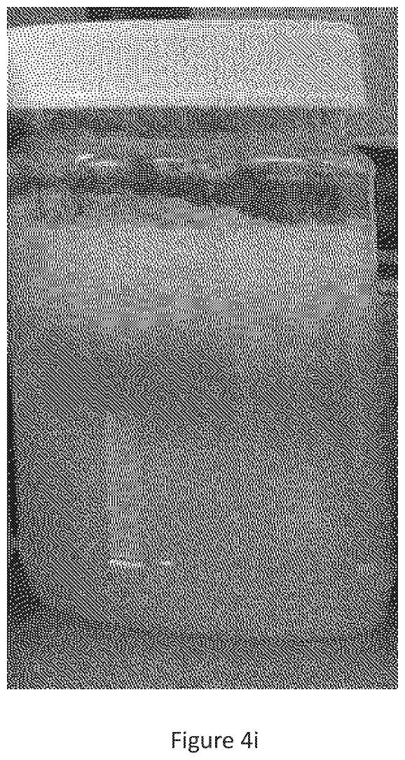

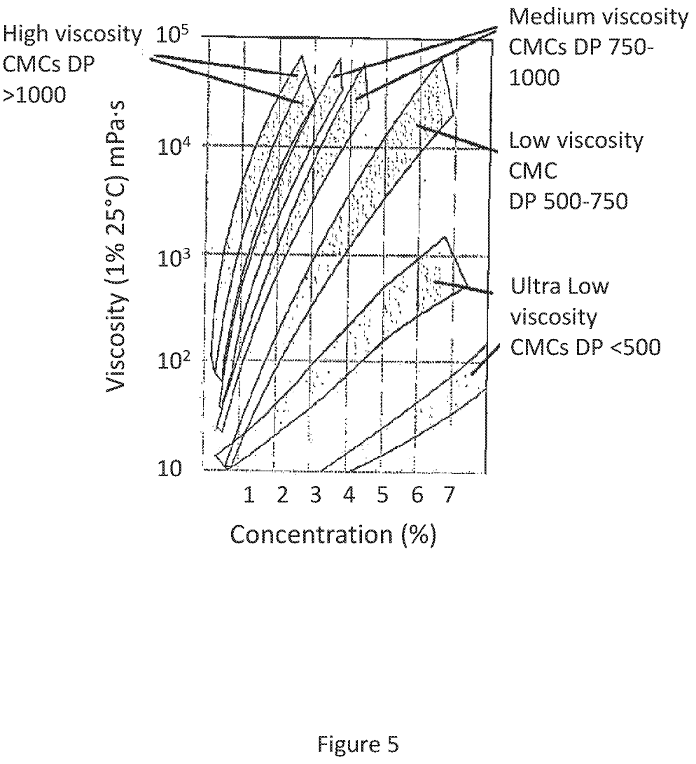

Comparative Example 1 shows the results obtained when a food grade sodium CMC (2% solution 30 mPa·s) was used in the method of JP2007. As per the teachings of JP2007, the CMC was first dissolved in water and stirred at 2000 rpm. However, contrary to the teachings of JP2007, the solution had to be mixed for 7 minutes at 4000 rpm in order to sufficiently hydrate the CMC, as mixing only at 2000 rpm provided a non-homogenous, lumpy solution. Then, xanthan gum was added, and the solution was stirred for a further 1 minute at 2000 rpm. In contrast to the viscosity of 1364 mPa·s reported in JP2007, the composition using a food grade CMC had an apparent viscosity of 8,000 mPa·s after allowing time for the gums to hydrate overnight as the product made was extremely lumpy. The composition was pourable but was thick and resisted flow. When used in a pump apparatus such as that described in the present application, the composition deteriorated when dispensed, indicating that the composition has a poor resistance to shear and is not suitable for bulk storage and delivery. In particular, the test was stopped at the 1 minute mark as lumps were observed, and the solution was hydrated for a further 2 minutes. The teachings of JP2007 indicate that the composition rapidly dispersed and expressed its viscosity. However, when the method was reproduced using a food grade CMC, the composition dispersed and expressed its viscosity very slowly. Furthermore, when mixed with water in a 1:5 ratio, the resulting solution had an apparent viscosity of 300 mPa·s, rather than the reported 3496 mPa·s. The thickened water solution had poor clarity (47.6% transmittance at 650 nm) and was cloudy and lumpy, as shown in FIG. 3b. The thickened water solution also showed poor stability, separating readily within 24 hours (FIG. 4b).

Comparative Example 2 shows the results obtained when a food grade sodium CMC (2% solution 50 mPa·s) was used in the method of JP2007. As per the teachings of JP2007, the CMC was first dissolved in water and stirred at 2000 rpm for 5 minutes. Similarly to Comparative Example 1, a further 2 minutes of stirring at 4000 rpm was required to hydrate the CMC. Then, xanthan gum was added, and the solution was stirred for a further 1 minute at 2000 rpm. Contrary to the teachings of JP2007, a further 6 minutes was required to incorporate the xanthan gum. In contrast to the viscosity of 1364 mPa·s reported in JP2007, the composition using this food grade CMC had an apparent viscosity of 30,000 mPa·s. The composition was extremely thick and not pourable or flowable. When used in a pump apparatus such as that described in the present application, the composition deteriorated when dispensed, indicating that the composition has a poor resistance to shear and is not suitable for bulk storage and delivery. The teachings of JP2007 indicate that the composition rapidly dispersed and expressed its viscosity. However, when reproduced using this food grade CMC, the composition was not dispersible. Furthermore, when mixed with water in a 1:5 ratio, the resulting solution had an apparent viscosity of 800 mPa·s, rather than the reported 3496 mPa·s. This thickened water solution also had moderate clarity (71.7% transmittance at 650 nm) and contained undispersed, cloudy lumps of the thickener composition, as shown in FIG. 3c. The thickened water solution also showed poor stability, separating readily within 24 hours (FIG. 4c).

Comparative Example 3 shows the results obtained when a combination of food grade sodium CMCs (8:2 ratio of 2% solution 30 mPa·s and 2% solution 50 mPa·s) were used in the method of JP2007. As per the teachings of JP2007, the CMCs were first dissolved in water and stirred at 2000 rpm for 5 minutes. However, this method did not sufficiently incorporate the CMC, so the stirring speed was increased to 4000 rpm for 5 minutes. Then, xanthan gum was added and the solution was stirred for a further 1 minute at 2000 rpm. In contrast to the viscosity of 1364 mPa·s reported in JP2007 (for a composition containing a single CMC), the composition using this combination of food grade CMCs had an apparent viscosity of 20 000 mPa·s. The composition was thick and showed limited pourability and flowability. When used in a pump apparatus such as that described in the present application, the composition deteriorated when dispensed, indicating that the composition has a poor resistance to shear and is not suitable for bulk storage and delivery. The teachings of JP2007 indicate that the composition rapidly dispersed and expressed its viscosity. However, when reproduced using this combination of food grade CMCs, the composition was not dispersible. Furthermore, when mixed with water in a 1:5 ratio, the resulting solution had an apparent viscosity of 150 mPa·s, rather than the reported 3496 mPa·s (for a composition with 1 CMC). This thickened water solution also had poor clarity (64.3% transmittance at 650 nm) and contained undispersed, cloudy lumps of the thickener composition, as shown in FIG. 3d. The thickened water solution also showed poor stability, separating readily within 24 hours (FIG. 4d).

Comparative Example 4 shows the results obtained when a combination of CMCs were used in the method of JP2007, with the additional step of hydrolysis prior to addition of the gum. The Applicant found that despite hydrolysing the viscosity inhibitor during the method of JP2007, they were unable to arrive at a composition with the advantageous qualities of the claimed invention. The food grade CMCs were first dissolved in water and stirred at 2000 rpm for 5 minutes. Then, the solution was hydrolysed at 90° C. for 12 hours Then, xanthan gum was added and the solution was stirred for a further 1 minute at 2000 rpm. The composition had an apparent viscosity of 4,000 mPa·s, but showed poor pourability and flowability. When used in a pump apparatus such as that described in the present application, the composition deteriorated when dispensed, indicating that the composition has a poor resistance to shear and is not suitable for bulk storage and delivery. When mixed with water in a 1:5 ratio, the resulting solution had an apparent viscosity of 3650 mPa·s but showed very poor dispersibility and did not form a homogenous mixture (FIG. 4e). This thickened water solution had moderate clarity (72.4% transmittance at 650 nm) and contained undispersed, cloudy lumps of the thickener composition, as shown in FIG. 3e.

Comparative Example 5 shows the results obtained when a xanthan gum (1% in 1% KCl 1300-1700 cPs, spindle #3 60 rpm) was used in the method of JP2007 without first dissolving sodium CMC. As per the teachings of JP2007, the xanthan gum was added and the solution was stirred for 5 minutes at 2000 rpm. As with above, the stirring speed was increased to 4000 rpm to sufficiently hydrate the CMC. Similar to the viscosity of 16,300 mPa·s reported in JP2007, the composition using xanthan gum had an apparent viscosity of 15400 mPa·s. The composition was extremely thick and not pourable or flowable. When used in a pump apparatus such as that described in the present application, the composition deteriorated when dispensed, indicating that the composition has a poor resistance to shear and is not suitable for bulk storage and delivery. In agreement with the teachings of JP2007, this composition was not dispersible. In contrast to the teachings of JP2007, when mixed with water in a 1:5 ratio, the resulting solution had an apparent viscosity of 2250 mPa·s, rather than the reported 242 mPa·s. The thickened water solution had high clarity (83.9% transmittance at 650 nm) but contained lumps of the thickener composition, as shown in FIG. 3f. Furthermore, this thickened water solution showed very poor dispersibility and did not form a homogenous mixture (FIG. 4f).

The following Comparative Examples (6-7) demonstrate the effect of incorporating the CMC and xanthan gum into the solution simultaneously, rather than through sequential addition. These examples demonstrate that the order in which the ingredients are added has a significant impact on the properties of the composition.

Comparative Example 6 shows the results obtained when a food grade sodium CMC (2% solution 30 mPa·s) was combined with xanthan gum (1% in 1% KCl 1300-1700 cPs, spindle #3 60 rpm) and used in the method of JP2007. The CMC and xanthan gums were first blended, then dissolved in water and stirred at 2,000 rpm for 1 minute. In contrast to the viscosity of 4,780 mPa·s reported in JP2007, the composition using this food grade CMC had an apparent viscosity of 15,000 mPa·s. The composition was thick and showed limited pourability and flowability. When used in a pump apparatus such as that described in the present application, the composition deteriorated when dispensed, indicating that the composition has a poor resistance to shear and is not suitable for bulk storage and delivery. These data agree with the teachings of JP2007 that the composition dispersed and expressed its viscosity very slowly. When mixed with water in a 1:5 ratio, the resulting solution had an apparent viscosity of 170 mPa·s, rather than the reported 3044 mPa·s. This thickened water solution also had high clarity (80.3% transmittance at 650 nm) and contained undispersed, cloudy lumps of the thickener composition, as shown in FIG. 3g. Furthermore, the thickened water solution showed very poor dispersibility, did not form a homogenous mixture and separated readily within 24 hours (FIG. 4g).

Comparative Example 7 shows the results obtained when a food grade sodium CMC (2% solution 50 mPa·s) was combined with xanthan gum (1% in 1% KCl 1300-1700 cPs, spindle #3 60 rpm) and used in the method of JP2007. The CMC and xanthan gums were first blended, then dissolved in water and stirred at 2,000 rpm for 1 minute. In contrast to the viscosity of 4,780 mPa·s reported in JP2007, the composition using this food grade CMC had an apparent viscosity of 17,500 mPa·s. The composition was thick and showed limited pourability and flowability. When used in a pump apparatus such as that described in the present application, the composition deteriorated when dispensed, indicating that the composition has a poor resistance to shear and is not suitable for bulk storage and delivery. When mixed with water in a 1:5 ratio, the resulting solution had an apparent viscosity of 300 mPa·s. This thickened water solution also had poor clarity (47.8% transmittance at 650 nm) and contained undispersed, cloudy lumps of the thickener composition, as shown in FIG. 3h. The thickened water solution also showed poor stability, separating readily within 24 hours (FIG. 4h).

Comparative Example 8 shows the results obtained when a combination of food grade CMCs was used in the method of JP2007. As per the teachings of JP2007, the CMCs were first dissolved in water and stirred at 2000 rpm for 5 minutes. Then, xanthan gum was added, and the solution was stirred for a further 1 minute at 2000 rpm. The composition had an apparent viscosity of 7,000 mPa·s, but showed poor pourability and flowability. When used in a pump apparatus such as that described in the present application, the composition deteriorated when dispensed, indicating that the composition has a poor resistance to shear and is not suitable for bulk storage and delivery. When mixed with water in a 1:5 ratio, the resulting solution had an apparent viscosity of 400 mPa·s. This solution had high clarity (98.4% transmittance at 650 nm) but contained undispersed, cloudy lumps of the thickener composition, as shown in FIG. 3i. The solution also showed poor stability, separating readily within 24 hours (FIG. 4i).

As can be seen from the above Comparative Examples, repeating the method of the prior art with food grade materials does not result in a thickener that is flowable, pumpable, homogenous (i.e., does not contain lumps or domains of undispersed gum/polysaccharide/food grade thickener), dispersible into an aqueous liquid so that thickened liquid is homogenous, has sufficient speed of hydration so the peak viscosity is reached within a short time frame (i.e., around 30-60 sec) at low shear (i.e., 80-160 BPM with a fork/spoon), has ability to withstand shear on delivery with a food grade pump, does not separate over time, is clear (no colour), and has little or no odour or taste. In all of the above cases in relation to the prior art, the resulting thickening compositions lack one or more of these desirable properties, and therefore are not suitable for use as thickening agents for use with patients with swallowing disorders.

Without wishing to be bound by any theory, the inventors hypothesise that the above results are due to the use solely of food grades of sodium CMC and not industrial grades which are known to have lower apparent viscosities. Specifically, the lowest food grade that could be sourced in commercial quantities is 10-20 mPa·s for a 2% solution. In contrast, JP2007 claims to use sodium CMC with a viscosity of 18 mPa·s (for a 10% solution). The inventors hypothesise that this is a significant contributing factor and that this is an industrial purified grade and therefore JP2007 has no applicability in modifying liquids for treatment of a swallowing disorder. As shown in Table 3, use of food grade sodium CMC in the method of JP2007 resulted in concentrate viscosities that are too high to allow a flowable, pumpable liquid that will readily disperse in our application. When the teachings of JP2007 are combined with a hydrolysis step (Comparative Example 4), the resulting composition was still not able to achieve the dispersibility, flowability, pourability and pumpability of the claimed invention.

In contrast, in preferred embodiments, the claimed invention has each of the aforementioned properties. For example, the food grade thickener of Example 4 has an apparent viscosity of 4,000 mPa·s, and is homogenous, flowable and pourable. When dissolved in a 1:5 ratio with water, the thickener disperses quickly and expresses its viscosity rapidly (within about 30 seconds), thickening the solution to 3000 mPa·s. The thickened water is homogenous and has a high clarity (84.7% transmittance at 650 nm) and does not deteriorate when dispensed through the pump apparatus, indicating a resistance to shear and suitability for bulk storage and delivery.

The data shown herein in relation to the method of the claimed invention, clearly shows that a food grade thickener that achieves the objectives of: food grade, pumpable, pourable, clear. It has further been shown herein that repeating the methods of JP2007 with food grade polysaccharides did not achieve these objectives. Therefore, it has been shown herein that JP2007 uses industrial grade polysaccharides to achieve the results claimed therein, and it will be appreciated that industrial grade polysaccharides are not fit for human consumption. It will be appreciated that the unique combination of employing a first polysaccharide and a second polysaccharide with a hydrolysis step, followed by addition of a gum to the hydrolysed mixture under conditions such that the gum only partially expresses its viscosity, and thereby forms a food grade thickener. It has been shown herein that the resulting food grade thickener has certain cohesion and adhesion properties that make it particularly suitable for delivery via a pumping apparatus and is flowable, pumpable, dispersible, clear and/or clear in the target foodstuff, homogenous and/or homogenous in the target foodstuff, and has little or no odour or taste and/or imparts little or no odour or taste in the target foodstuff.

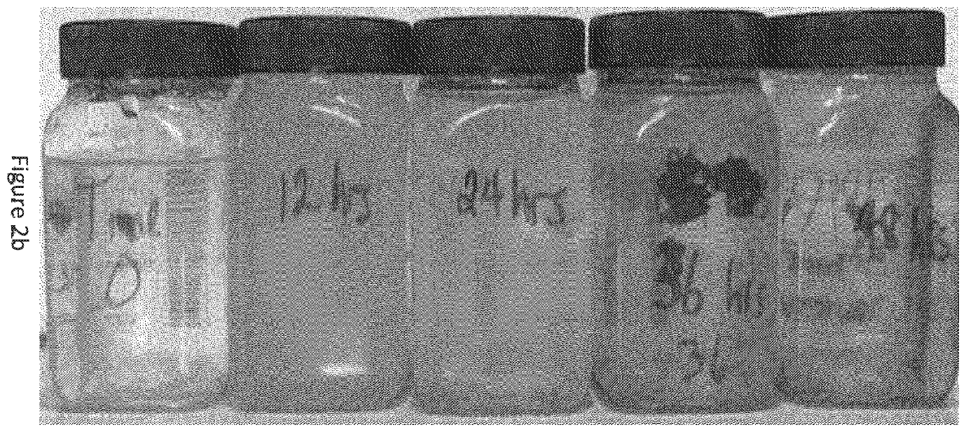

TABLE 3
Summary of comparative data.

| | Data | Comparative examples | | | | | | | |
|---|---|---|---|---|---|---|---|---|---|
| Results | source | 1 | 2 | 3 | 4 | 5 | 6 | 7 | 8 |
| Brief description | | 100% | 100% | 80% food | 80% food | 100% | 50:50 blend | 50:50 blend | 80% food |
| | | Food | Food | grade | grade | Xanthan | of xanthan | of xanthan | grade |
| | | grade | Grade | Na-CMC | Na-CMC | formulation | and | and | Na-CMC |
| | | Na-CMC | Na-CMC | 2% solution | 2% solution | made using | Na-CMC | Na-CMC | 2% solution |
| | | 2% solution | 2% solution | 30 mPa · s + | 30 mPa · s + | JP2007 | 2% solution | 2% solution | 30 mPa · s + |
| | | 30 mPa · s) | 50 mPa · s) | 20% Food | 20% Food | method | 30 mPa · s) | 50 mPa · s) | 20% Food |
| | | formulation | formulation | Grade | Grade | | formulation | formulation | Grade |
| | | made using | made using | NACMC | NaCMC | | made using | made using | NaCMC |
| | | JP2007 | JP2007 | 2% solution | 2% solution | | JP2007 | JP2007 | 2% solution |
| | | method | method | 50 mPa · s) | 50 mPa · s) | | method | method | 50 mPa · s) |
| | | | | formulation | formulated | | | | formulated |
| | | | | made using | using | | | | using |
| | | | | JP2007 | JP2007 | | | | JP2007 |
| | | | | method | teaching | | | | method |
| | | | | | (modified | | | | exactly |
| | | | | | dispermix | | | | |
| | | | | | time and | | | | |
| | | | | | speed as | | | | |
| | | | | | appropriate) | | | | |
| | | | | | combined | | | | |
| | | | | | with acid | | | | |
| | | | | | heat | | | | |
| | | | | | hydrolysis | | | | |
| | | | | | (90° C./12 | | | | |
| | | | | | hours) | | | | |
| JP2007 counter- | | Example 1 | Example 1 | Example 1 | NA | Compara- | Compara- | Compara- | NA |
| part experiment | | | | | | tive 1 | tive 3 | tive 3 | |
| Food grade | Comparative | 8,000.00 | 30,000.00 | 20,000.00 | 4,000.00 | 15,400.00 | 15,000.00 | 7,500.00 | 7000 |
| thickener | experiments | | | | | | | | |
| viscosity¹ | Data from | 1,364.00 | 1,364.00 | 1,364.00 | N/A | 16,300.00 | 4,780.00 | 4,780.00 | NA |
| (mPa · s) | JP2007² | | | | | | | | |
| 20% solution | Comparative | 300.00 | 800.00 | 150.00 | 3,650.00 | 2,250.00 | 170.00 | 300.00 | 400 |
| of food grade | experiments³ | | | | | | | | |
| thickener | Data from | 3,496.00 | 3,496.00 | 3,496.00 | NA | 242.00 | 3,044.00 | NA | NA |
| in water | JP2007⁴ | | | | | | | | |
| Viscosity¹ | | | | | | | | | |
| (mPa · s) | | | | | | | | | |
| Dispersibility | Comparative | dispersed | non- | non- | dispersed | non- | dispersed | non- | non- |
| | experiments | slowly and | dispersable | dispersable | slowly and | dispersable | slowly and | dispersable | dispersable |
| | | viscosity | | | viscosity | | viscosity | | |
| | | development | | | development | | development | | |
| | | eventually | | | eventually | | eventually | | |
| | Data from | dispersed | dispersed | dispersed | N/A | non- | dispersed | dispersed | NA |
| | JP2007 | rapidly and | rapidly and | rapidly and | | dispersable | slowly and | slowly and | |
| | | viscosity | viscosity | viscosity | | | viscosity | viscosity | |
| | | developed | developed | developed | | | development | development | |
| | | | | | | | eventually | eventually | |
| Clarity of 20% | | 47.6 | 71.7 | 64.3 | 72.4 | 83.9 | 80.3 | 47.8 | 98.4 |
| food grade | | | | | | | | | |
| thickener solution | | | | | | | | | |
| (% transmittance | | | | | | | | | |
| at 650 nm, 1 cm | | | | | | | | | |
| path length) | | | | | | | | | |
| Homogeneity of | | Does not | Does not | Does not | homogenous, | Does not | homogenous, | Does not | Does not |
| food grade | | disperse, | disperse, | disperse, | dispersion | disperse, | dispersion | disperse, | disperse, |
| thickener | | lumpy | lumpy | lumpy | no lumps | lumpy | no lumps | lumpy | lumpy |
| Homogeneity of | Images | FIG. 2 b | FIG. 2 c | FIG. 2 d | FIG. 2 e | FIG. 2 f | FIG. 2 g | FIG. 2 h | FIG. 2 i |
| 20% food grade | | | | | | | | | |
| thickener solution | | | | | | | | | |
| Pourability/Flow- | | thick, | not | thick, | thick, | not | thick, | thick, | thick, |
| ability of the | | pourable | pourable, | pourable | pourable | pourable, | pourable | pourable | pourable |
| food grade | | with | extremely | with | with | extremely | with | with | with |
| thickener | | resistance | thick | resistance | resistance | thick | resistance | resistance | resistance |
| | | to flow | | to flow | to flow | | to flow | to flow | to flow |
| Food grade | | Separates | Separates | Separates | non | non | Separates | Separates | Separates |
| thickener | | readily | readily | readily | homogenous | homogenous | readily | readily | readily |
| Separation/homo- | | (<24 | (<24 | (<24 | (lumps) | (lumps) | (<24 | (<24 | (<24 |
| geneity | | hours) | hours) | hours) | | | hours) | hours) | hours) |
| | | | | | | | and non | | |
| | | | | | | | homogenous | | |
| | | | | | | | (lumps) | | |
| Pumpability/resis- | | not | not | not | not | not | not | not | not |
| tance to shear | | dispersible. | dispersible. | dispersible. | dispersible. | dispersible. | dispersible. | dispersible. | dispersible |
| on pumping/dis- | | FIG. 3b | FIG. 3c | FIG. 3d | FIG. 3e | FIG. 3f | FIG. 3g | FIG. 3h | FIG. 3i |
| persibility throw | | | | | | | | | |
| Pumpability/resis- | | | Highly | | | Composition | | After being | |
| tance to shear | | | viscous | | | is thick and | | pumped, | |
| on pumping/dis- | | | after being | | | does not | | composition | |
| persibility throw | | | pumped, | | | disperse in | | is thick and | |
| | | | very poor | | | the solution, | | lumpy and | |
| | | | dispersion | | | slightly | | does not | |
| | | | after | | | cloudy and | | disperse in | |
| | | | mixing, | | | lumpy on | | the solution | |
| | | | cloudy | | | the bottom | | | |
| | | | lumps in | | | | | | |
| | | | the bottom. | | | | | | |

1. Measured using a Brookfield rotational viscometer, 20 rpm (Spindle #5 or #6 depending on viscosity) at 25° C.
2. Measured using a B-type viscometer.
3. Stirred at 75 rpm 30 seconds.
4. Stirred at 4 rpm 15 seconds.

Experiments and Tests

### Test A1: Method of Manufacture

The following general method is used for manufacture of a thickener composition, and used in respect of the Tests A2 and A3 below.
A polysaccharide of molecular weight less than 500 000 is dissolved in water with good agitation.
Other additives such as food acids, colours, flavours and preservatives are added to the solution and mixed to dissolution.
The solution is heated to between 60 to 90° C.
A thickening agent is added to the hot solution and mixed to disperse.
The resultant solution is then hot filled into a food package at a minimum temperature of 60° C.

### Test A2: Composition 1

| | |
|---|---|
| Alternan | 10% |
| Guar Gum | 10% |
| Water | 79.7% |
| Citric Acid | 0.3% |
| Potassium Sorbate | 0.07% |

Using the method described in Test A1, this composition is a flowable liquid of 2500 cP. When 5 g of this liquid is added to 100 millilitres of orange juice and mixed well to disperse, the resulting solution thickens quickly to a viscosity of 250 cP. The composition is stable and has a shelf life of 10 months when stored at room temperature.

### Test A3: Composition 2

| | |
|---|---|
| Sodium CMC (MW 3200) | 6% |
| Xanthan Gum | 8% |
| Water | 85.7% |
| GDL | 0.23% |
| Potassium Sorbate | 0.07% |

Using the method described in Test A1, this composition is a flowable liquid of 1700 cP. When 10 g of this liquid is added to 100 millilitres of water and mixed well to disperse, the resulting solution thickens quickly to a viscosity of 430 cP. The composition is stable and has a shelf life of 12 months when stored at room temperature.

### Test A4: Composition 3

An arabinogalactan fragment solution is obtained from the acid hydrolysis (at 80° C.) of polysaccharides extracted from Acacia trees. A 16% dry solids solution of the arabinoglalactan fragment solution is obtained by concentration (removal of water) giving a solution of viscosity 55 cP. 9 wt. % xanthan gum is then added to produce a thickening concentrate of 1100 cP viscosity. The solution is acidified with sodium acid bisulfite to pH 4.5 and 1000 ppm of benzoic acid added as a preservative. The solution is then heated to 78° C. and hot filled in plastic bottles. When 15 g of this thickening concentrate is added to 100 millilitres of miso soup and mixed well to disperse, the resulting solution thickens quickly to a viscosity of 620 cP. The composition is stable and has a shelf life of 12 months when stored at room temperature.

Test A5: Composition 4

A 7 wt % solution of xanthan gum is enzyme hydrolysed using xanthan depolymerase (endo-beta-1,4-glucanase) enzyme. The resulting solution has a viscosity of 83 cP. The enzyme hydrolysate is filtered and purified to remove suspended materials and contaminants and then 7 wt. %, of native, unhydrolysed xanthan gum powder is added to the solution to produce a thickening concentrate of 1650 cP viscosity. The solution is acidified with citric acid to pH 4.5 and 1000ppm of benzoic acid added as a preservative. The solution is then heated to 75° C. and hot filled in plastic bottles. When of this thickening concentrate is added to 100 millilitres of hot green tea and mixed well to disperse, the resulting solution thickens quickly to a viscosity of 110 cP. The composition is stable and has a shelf life of 9 months when stored at room temperature.

### Test B: Method of Manufacture

### Test B1

4 parts of medium viscosity sodium carboxymethylcellulose (degree of polymerisation 750-1000) were added to 96 parts water and mixed to disperse the powder. The viscosity of the solution was measured as 977 mPa·s (Rotor 3, 30 rpm using NDJ-5S Digital Rotary Viscometer). The solution was acidified to pH 4.2 using glucono delta lactone and heated to 90° C. for 120 minutes. The solution was cooled to room temperature and the viscosity of the acid hydrolysed solution measured, which was found to be 312 mPa·s (Rotor 3, 30 rpm using NDJ-5S Digital Rotary Viscometer). Greater than a 3-fold reduction in viscosity was achieved by the acid hydrolysis.

5 parts xanthan gum was added to the cooled acid hydrolysed solution of sodium carboxymethylcellulose with gentle mixing. This produced a flowable food grade thickener of 2160 mPa·s (Rotor 1, 30rpm using NDJ-5S Digital Rotary Viscometer) which is 9 times less viscous than a 5% xanthan gum solution in water, proving the effectiveness of the acid hydrolysed sodium carboxymethylcellulose in inhibiting the viscosity of xanthan gum (see FIG. 6).

When 8 parts of the xanthan gum containing solution made above was added to 92 parts water and gently mixed by hand, the liquid quickly dispersed and increased the viscosity of the water to 727 mPa·s (Rotor 2, 30 rpm, using NDJ-5S Digital Rotary Viscometer) within 30 seconds of mixing.

This formulation was made shelf stable with a shelf life of at least 12 months by adding 700 ppm of potassium sorbate (or other permitted food preservative) and hot filling into a hermetically sealed container at 80° C.

### Test B2

6 parts of sodium alginate was added to 94 parts water and mixed to disperse the powder. The viscosity of the solution was measured as 3948 mPa·s (Rotor 3, 30 rpm using NDJ-5S Digital Rotary Viscometer). The solution was acidified to pH 4.4 using citric acid and heated to 90° C. for 60 minutes. The solution was cooled to room temperature and the viscosity of the acid hydrolysed solution was measured, which was found to be 63 mPa·s (Rotor 1, 60 rpm using NDJ-5S Digital Rotary Viscometer). Greater than a 63-fold reduction in viscosity was achieved by the acid hydrolysis.

5 parts xanthan gum was added to the cooled acid hydrolysed solution of sodium alginate with gentle mixing. This produced a flowable food grade thickener of 3948 mPa·s (Rotor 3, 30 rpm using NDJ-5S Digital Rotary Viscometer) which is almost 5 times less viscous than a 5% xanthan gum solution in water, proving the effectiveness of the acid hydrolysed sodium alginate in inhibiting the viscosity of xanthan gum (see FIG. 7).

When 8 parts of the xanthan gum containing solution made above was added to 92 parts water and gently mixed by hand, the liquid quickly dispersed and increased the viscosity of the water to 768 mPa·s (Rotor 2, 30rpm, using NDJ-5S Digital Rotary Viscometer) within 30 seconds of mixing.

This formulation was made shelf stable with a shelf life of at least 12 months by adding 700 ppm of potassium sorbate (or other permitted food preservative) and hot filling into a hermetically sealed container at 80° C.

### Test B3

2 parts of xanthan gum was added to 98 parts water and mixed to disperse the powder. The viscosity of the solution was measured as 3827 mPa·s (Rotor 3, 30 rpm using NDJ-Digital Rotary Viscometer). Xanthan depolymerase was added at a concentration of 3.6×10-4 IU/mL with 0.4 mM MgSO₄ and 0.03 mM MnSO₄ in 0.05 M sodium acetate buffer (pH 5.4) and incubated at 32-34° C. for 20 minutes. The enzyme was deactivated by heating to 50° C., then the solution was cooled to room temperature and its viscosity was measured. Viscosity after enzyme hydrolysis was found to be 94 mPa·s (Rotor 1, 30 rpm using NDJ-5S Digital Rotary Viscometer). A 40-fold reduction in viscosity was achieved by the enzyme hydrolysis.

5 parts xanthan gum was added to the cooled enzyme hydrolysed solution of xanthan gum with gentle mixing. This produced a flowable food grade thickener of 2455 mPa·s (Rotor 2, 30 rpm using NDJ-5S Digital Rotary Viscometer) which is 8 times less viscous than a 5% xanthan gum solution in water, proving the effectiveness of the enzyme hydrolysed xanthan gum in inhibiting the viscosity of unhydrolyzed xanthan gum (see FIG. 8).

When 8 parts of the xanthan gum containing solution made above was added to 92 parts water and gently mixed by hand, the liquid quickly dispersed and increased the viscosity of the water to 740 mPa·s (Rotor 2, 30 rpm, using NDJ-5S Digital Rotary Viscometer) within 30 seconds of mixing.

This formulation was made shelf stable with a shelf life of at least 12 months by adding w/w glucono delta-lactone (or other permitted food acid) and 700 ppm of potassium sorbate (or other permitted food preservative) and hot filling into a hermetically sealed container at 80° C.

### Test B4

2 parts of guar gum were added to 98 parts water and mixed to disperse the powder.

The viscosity of the solution was measured as 2870 mPa·s (Rotor 3, 30rpm using NDJ-5S Digital Rotary Viscometer). β-mannanase was added at a concentration of 8.3×10⁻⁴ IU/mL and phosphate buffer (pH 7.0) and incubated at 25° C. for 30 minutes. The enzyme was deactivated by heating to 90° C. then the solution was cooled to room temperature and the viscosity measured. Viscosity after enzyme hydrolysis=410 mPa·s (Rotor 1, 30 rpm using NDJ-5S Digital Rotary Viscometer). A 7-fold reduction in viscosity was achieved by the enzyme hydrolysis.

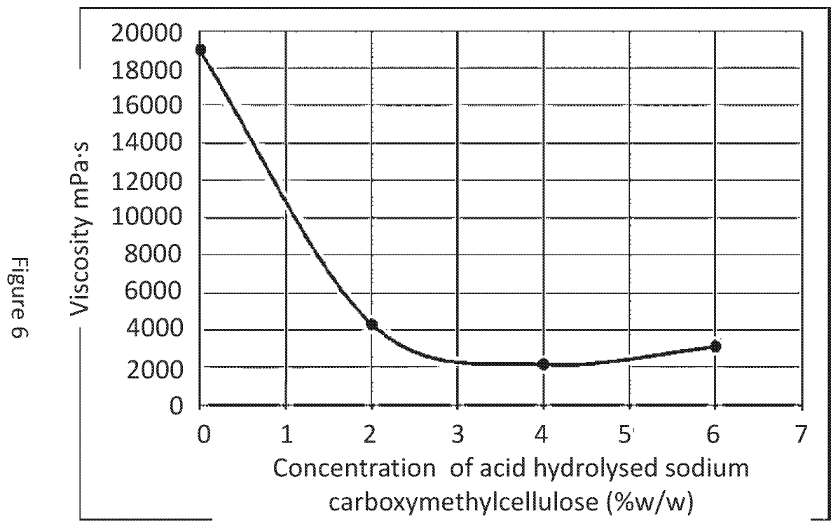

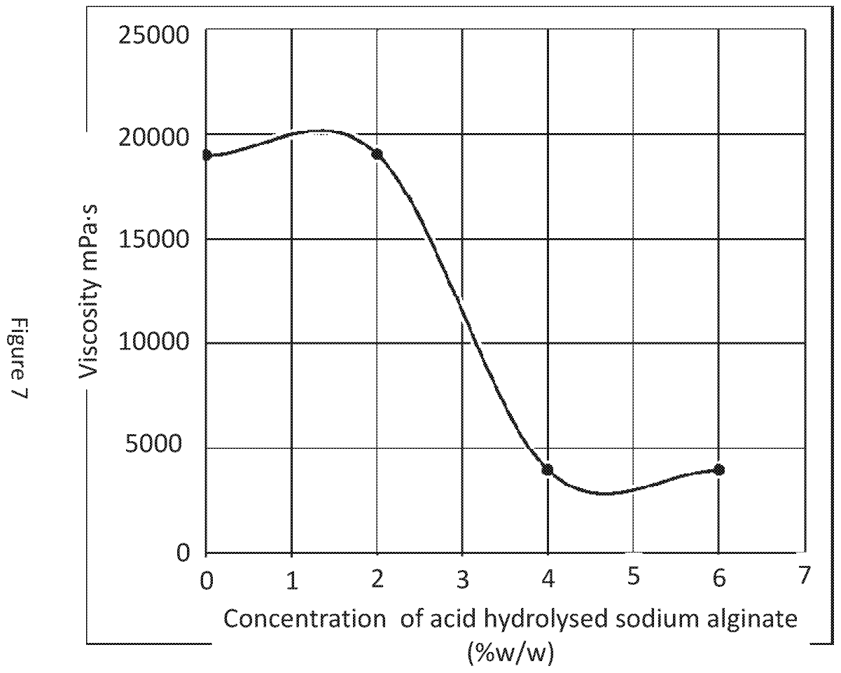

5 parts xanthan gum was added to the cooled enzyme hydrolysed solution of guar gum with gentle mixing. This produced a flowable food grade thickener of 3475 mPa·s (Rotor 2, 30 rpm using NDJ-5S Digital Rotary Viscometer) which is 5 times less viscous than a 5% xanthan gum solution in water, proving the effectiveness of the enzyme hydrolysed guar gum in inhibiting the viscosity of xanthan gum (see FIG. 9).

When 8 parts of the xanthan gum containing solution made above was added to 92 parts water and gently mixed by hand, the liquid quickly dispersed and increased the viscosity of the water to 790 mPa·s (Rotor 2, 30 rpm, using NDJ-5S Digital Rotary Viscometer) within 30 seconds of mixing.

This formulation was made shelf stable with a shelf life of at least 12 months by adding 0.6% w/w glucono delta-lactone (or other permitted food acid) and 700 ppm of potassium sorbate (or other permitted food preservative) and hot filling into a hermetically sealed container at 80° C.

### Test B5

4 parts of methyl ethyl cellulose with an average degree of polymerisation of 250 was added to 96 parts water and mixed to disperse the powder. The viscosity of the solution was measured as 95 mPa·s (Rotor 1, 30 rpm using NDJ-5S Digital Rotary Viscometer).

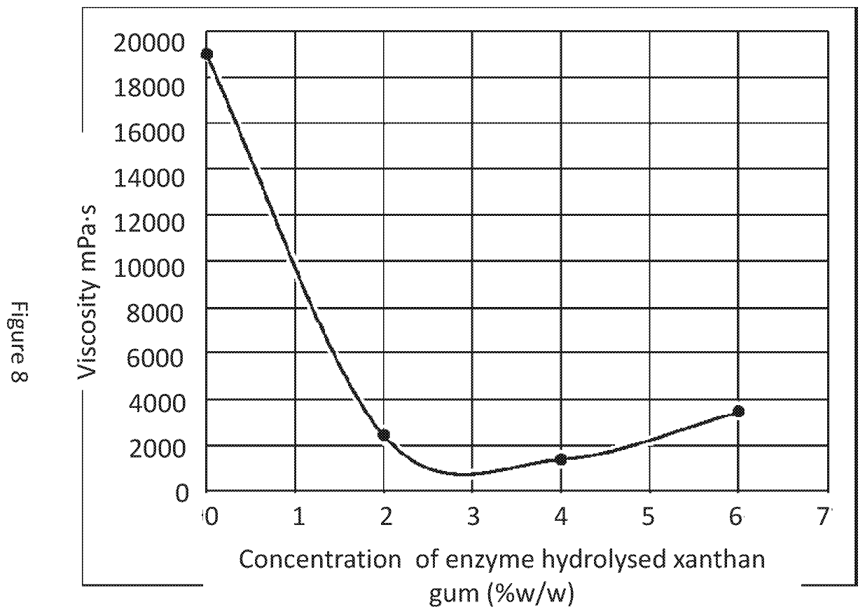

5 parts xanthan gum was added to the solution of methyl ethyl cellulose with gentle mixing. This produced a flowable food grade thickener of 3340 mPa·s (Rotor 3, 30rpm using NDJ-5S Digital Rotary Viscometer) which is about 6 times less viscous than a 5% xanthan gum solution in water proving the effectiveness of methyl ethyl cellulose with an average degree of polymerisation of 250 in inhibiting the viscosity of xanthan gum (see FIG. 10).

When 8 parts of the xanthan gum containing solution made above was added to 92

parts water and gently mixed by hand, the liquid quickly dispersed and increased the viscosity of the water to 740 mPa·s (Rotor 2, 30 rpm, using NDJ-5S Digital Rotary Viscometer) within 30 seconds of mixing.

This formulation was made shelf stable with a shelf life of at least 12 months by adding 0.6% w/w glucono delta-lactone (or other permitted food acid) and 700 ppm of potassium sorbate (or other permitted food preservative) and hot filling into a hermetically sealed container at 80° C.

### Test B6

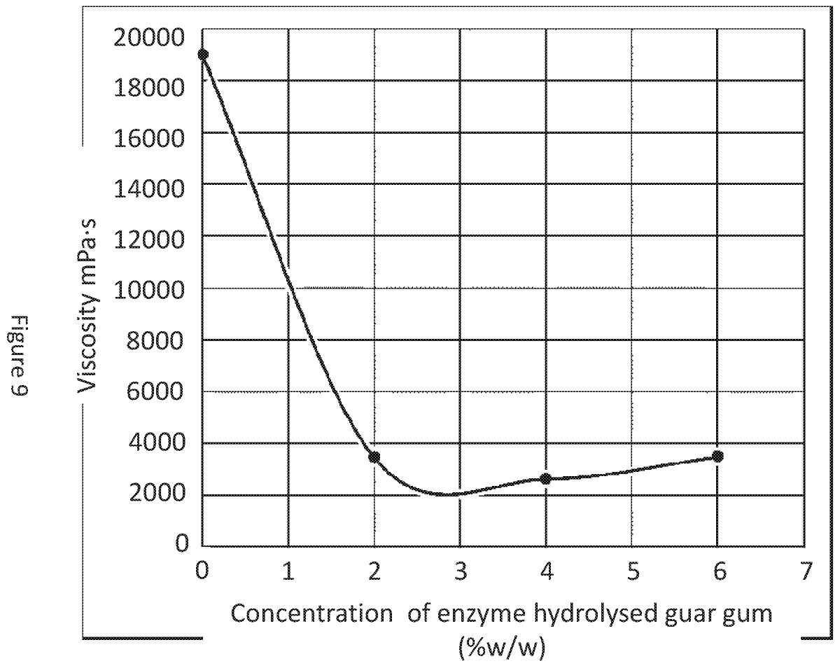

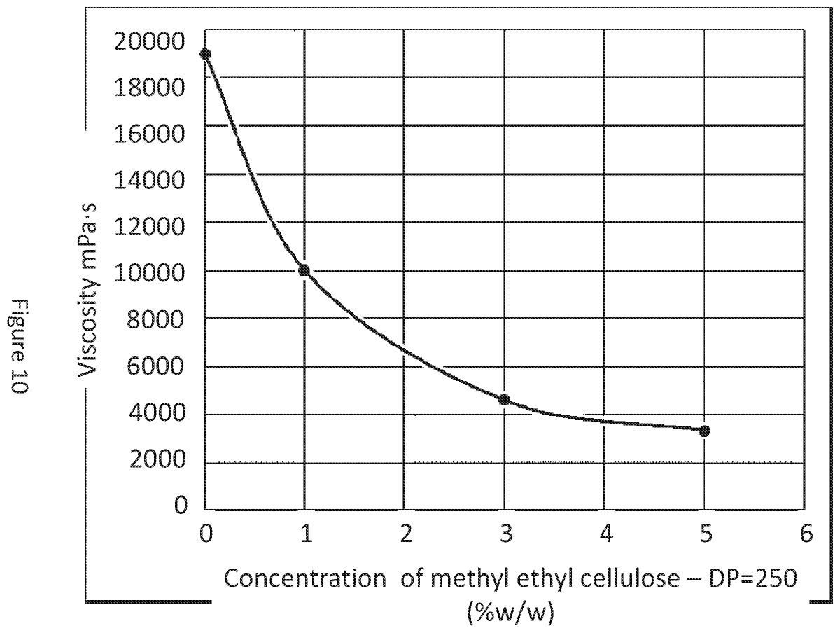

2.5 parts of sodium carboxymethylcellulose with an average degree of polymerisation of 120-150 was added to 97.5 parts water and mixed to disperse the powder. The viscosity of the solution was measured as 39 mPa·s (Rotor 1, 30 rpm using NDJ-5S Digital Rotary Viscometer). 4.5 parts xanthan gum was added to the solution of sodium carboxymethylcellulose with gentle mixing. This produced a flowable food grade thickener of 2450 mPa·s (Rotor 3, 60 rpm using NDJ-5S Digital Rotary Viscometer) which is about 8 times less viscous than a 5% xanthan gum solution in water proving the effectiveness of sodium carboxymethylcellulose with an average degree of polymerisation of 120-150 in inhibiting the viscosity of xanthan gum (see FIG. 11).

When 7 parts of the xanthan gum containing solution made above was added to 93 parts water and gently mixed by hand, the liquid quickly dispersed and increased the viscosity of the water to 747 mPa·s (Rotor 2, 30 rpm, using NDJ-5S Digital Rotary Viscometer) within 30 seconds of mixing.

This formulation was made shelf stable with a shelf life of at least 12 months by adding 0.6% w/w glucono delta-lactone (or other permitted food acid) and 700 ppm of potassium sorbate (or other permitted food preservative) and hot filling into a hermetically sealed container at 80° C.

### Test B7

The following formulation gives an example of how the three types of low viscosity, highly soluble, polysaccharides mentioned above can be combined to produce a flowable viscosity-inhibited composition:
Water—91% w/w
Xanthan gum (in its native unhydrolyzed form)—5% w/w
Xanthan gum (enzyme hydrolysed)—1% w/w
Sodium carboxymethyl cellulose (degree of polymerisation 120-150)—1.0% w/w
Sodium carboxymethyl cellulose (degree of polymerisation>500, acid hydrolysed)—0.5% w/w
Sodium alginate (acid hydrolysed)—1% w/w
Pectin (acid hydrolysed)—1% w/w

Method: 1 part of xanthan gum was added to 99 parts water and mixed to disperse the powder. Xanthan depolymerase was added at a concentration of 3.6×10-4 IU/mL with 0.4 mM MgSO₄ and 0.03 mM MnSO₄ in 0.05 M sodium acetate buffer (pH 5.4) and incubated at 32-34° C. for 20 minutes. The three gums to be acid hydrolysed (0.5 parts of sodium carboxymethyl cellulose-degree of polymerisation greater than 500; 1 part of sodium alginate; and 1 part pectin) were added under good shear mixing and acidified to pH 4.1 using glucono delta lactone. The solution was heated to 90° C. for 60 minutes (this heating step also deactivates the xanthan depolymerase enzyme). Finally,1 part sodium carboxymethyl cellulose (degree of polymerisation 120-150) was added under good shear mixing. The viscosity of the solution was measured as 76 mPa·s (Rotor 1, using NDJ-5S Digital Rotary Viscometer).

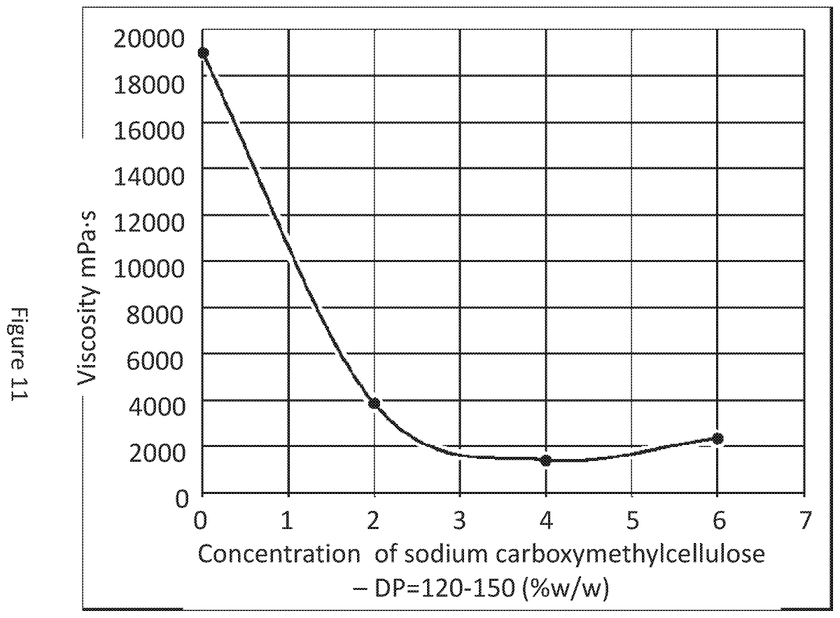

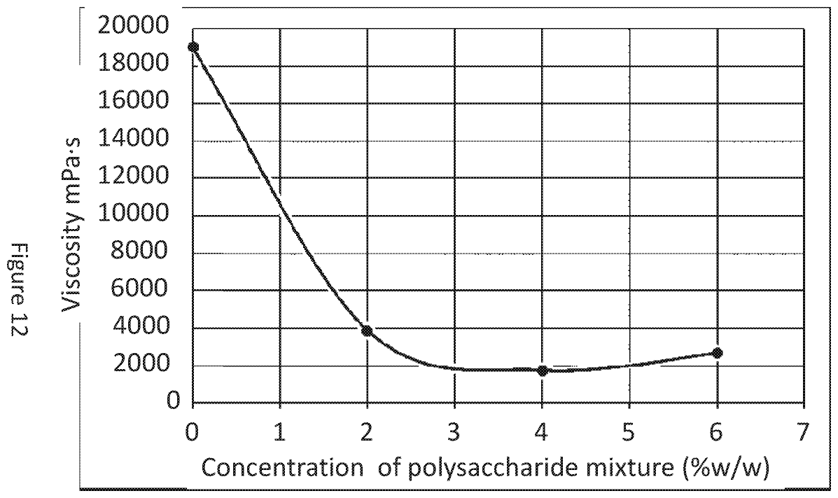

5 parts xanthan gum was added to the mixture of low viscosity, highly soluble polysaccharides prepared above with gentle mixing. This produced a flowable food grade thickener of 1740 mPa·s (Rotor 1, 30 rpm using NDJ-5S Digital Rotary Viscometer) which is 11 times less viscous than a 5% xanthan gum solution in water, proving the effectiveness of the mixture in inhibiting the viscosity of xanthan gum (see FIG. 12). The effect is similar and, in some cases, better than the individual low viscosity, highly soluble, polysaccharides when used singularly in Tests B1-6 above.

When 8 parts of the xanthan gum containing solution made above was added to 92 parts water and gently mixed by hand, the liquid quickly dispersed and increased the viscosity of the water to 705 mPa·s (Rotor 2, 30 rpm, using NDJ-5S Digital Rotary Viscometer) within 30 seconds of mixing.

This formulation was made shelf stable with a shelf life of at least 12 months by adding 0.6% w/w glucono delta-lactone (or other permitted food acid) and 700 ppm of potassium sorbate (or other permitted food preservative) and hot filling into a hermetically sealed container at 80° C.

### Test B8

The following formulation shows how the three types of low viscosity, highly soluble, polysaccharides mentioned above can be combined to produce a flowable viscosity-inhibited composition:
Water—80% w/w
Sodium alginate (in its native unhydrolyzed form)—8% w/w
Pectin (acid hydrolysed)—1% w/w
Hydroxypropyl methylcellulose (degree of polymerisation 200)—1% w/w
Guar gum (enzyme hydrolysed)—1% w/w

Method: 1 part of guar gum was added to 99 parts water and mixed to disperse the powder. β-mannanase was added at a concentration of 8.3×10-4 IU/mL and phosphate buffer (pH 7.0). The solution was incubated at 25° C. for 30 minutes. The pectin was then added to be acid hydrolysed (1 part) under good shear mixing and the solution acidified to pH 4.1 using tartaric acid. The solution was then heat to 90° C. for 60 minutes (this heating step also deactivated the 8-mannanase enzyme). Finally, under good shear mixing, 1-part hydroxypropyl methylcellulose (degree of polymerisation 200) was added. The viscosity of the solution was measured as 75 mPa·s (Rotor 1, 30 rpm using NDJ-5S Digital Rotary Viscometer).

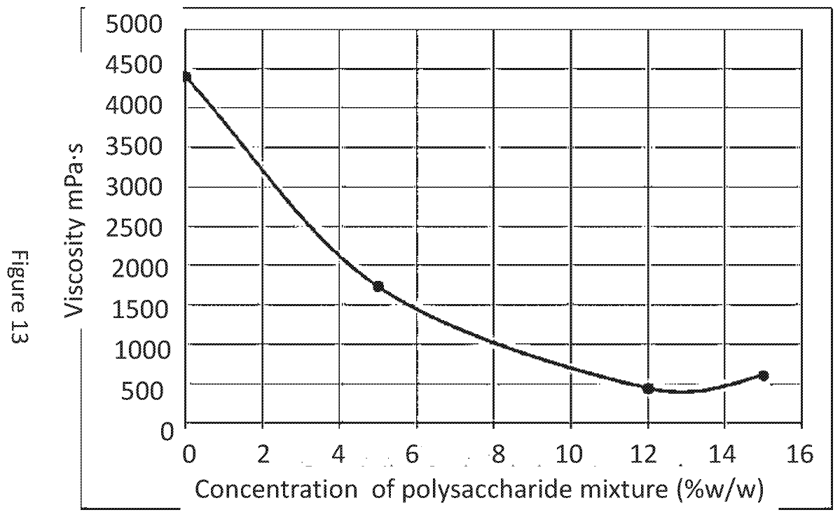

8 parts sodium alginate was added to the mixture of low viscosity, highly soluble polysaccharides prepared above with gentle mixing. This produced a flowable food grade thickener of 440 mPa·s (Rotor 1, 30 rpm using NDJ-5S Digital Rotary Viscometer) which is 10 times less viscous than an 8% sodium alginate solution in water, proving the effectiveness of the mixture in inhibiting the viscosity of sodium alginate (see FIG. 13).

When 12 parts of the sodium alginate gum containing solution made above was added to 88 parts water and gently mixed by hand, the liquid quickly dispersed and increased the viscosity of the water to 610 mPa·s (Rotor 2, 30 rpm, using NDJ-5S Digital Rotary Viscometer) within 30 seconds of mixing.

This formulation was made shelf stable with a shelf life of at least 12 months by adding 0.6% w/w glucono delta-lactone (or other permitted food acid) and 700 ppm of potassium sorbate (or other permitted food preservative) and hot filling into a hermetically sealed container at 80° C.

Although the invention has been described with reference to specific examples, it will be appreciated by those skilled in the art that the invention may be embodied in many other forms in particular features of any one of the various described examples may be provided in any combination in any of the other described examples. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein.

Other embodiments of the invention as described herein are defined in the following paragraphs:

1. A method for providing a food grade thickener, the method comprising the steps of:
providing an aqueous phase,
adding a polysaccharide to the aqueous phase thereby forming a gelled mixture,
hydrolysing the gelled mixture to reduce the viscosity of the gelled mixture, and
adding a gum to the hydrolysed gelled mixture under conditions such that the gum only partially expresses its viscosity, thereby forming the food grade thickener.
2. The method according to paragraph 1, wherein the polysaccharide is selected from the group consisting of: agar, alginic acid, carrageenan, guar gum, gum tragacanth, gum ghatti, microcrystalline cellulose, sodium carboxymethylcellulose, methyl cellulose, hydroxypropylmethylcellulose, hydroxyproylcellulose, methylethylcellulose, gum karaya, locust bean gum, tara gum, psyllium seed gum, quince seed gum, a pectin, furcellaran, gellan gum, konjac, sodium alginate, xanthan gum or a combination thereof.
3. The method according to any one of the preceding paragraphs, wherein the polysaccharide is added to the aqueous phase in a concentration of about 0.5 to 30 wt %.
4. The method according to any one of the preceding paragraphs, wherein the gelled mixture is hydrolysed at a temperature of about 50 to 95° C.
The method according to any one of the preceding paragraphs, wherein the gelled mixture is hydrolysed for a duration of about 2 to 72 hours.
6. The method according to any one of the preceding paragraphs, wherein the gelled mixture is acid hydrolysed to produce the hydrolysed gelled mixture.
7. The method according to any one of the preceding paragraphs, wherein the hydrolysed gelled mixture has a viscosity of between about 40-150 cP measured at 20° C. using a Brookfield viscometer #5 spindle at 10 RPM.
8. The method according to any one of the preceding paragraphs, wherein the gum is selected from the group consisting of: agar, alginic acid, carrageenan, guar gum, gum tragacanth, gum ghatti, microcrystalline cellulose, sodium carboxymethylcellulose, methyl cellulose, hydroxypropylmethylcellulose, hydroxyproylcellulose, methylethylcellulose, gum karaya, locust bean gum, tara gum, psyllium seed gum, quince seed gum, a pectin, furcellaran, gellan gum, konjac, sodium alginate, fenugreek gum, guar gum, tara gum and locust bean gum, xanthan gum and any combination thereof.
9. The method according to any one of the preceding paragraphs, wherein the gum is added in a concentration of about 2 to 30 wt %.
10. A food grade thickener when produced by the method according to any one of paragraphs 1 to 9.
11. The food grade thickener according to paragraph 10, wherein the food grade thickener is stable for at least six months.
12. The food grade thickener to any one of paragraphs 10 to 11, wherein the food grade thickener has a viscosity of about 500 to 10,000 cP measured at 20° C. using a Brookfield viscometer #3 spindle at 5 RPM.
13. The food grade thickener to any one of paragraphs 10 to 12, wherein the food grade thickener has a resistance to flow of greater than about 12 cm at 20° C. at 30 seconds measured using a Bostwick consistometer.
14. The food grade thickener to any one of paragraphs 10 to 13, wherein a 7 wt % solution of the food grade thickener and water has a transmittance of about >90% at 650 nm when measured using a lcm path length.
15. A method for increasing the viscosity of an aqueous liquid or aqueous liquid solid mixture foodstuff, the method comprising the step of adding to the foodstuff the food grade thickener according to any one of paragraphs 10 to 14.
16. The method according to paragraph 15, wherein the amount of food grade thickener that is added is about 1 to 30 wt %.
17. The method according to paragraph 15 or paragraph 16, wherein adding the food grade thickener to the foodstuff causes the viscosity of the foodstuff is increased to at least about 95 cP.
18. A method of treating a subject suffering from a mastication and/or deglutition disease, disorder or condition, the method comprising the step of administering a foodstuff to the subject, wherein the foodstuff comprises the food grade thickener according to any one of paragraphs to 14.
19. Use of the food grade thickener according to any one of paragraphs 10 to 14 in the manufacture of a medicament for the treatment or amelioration of a mastication and/or deglutition disease, disorder or condition.
20. A method of overcoming or ameliorating difficulties in swallowing in a patient in need of such treatment, comprising the step of thickening a food or beverage with the food grade thickener according to any one of paragraphs 10 to 14 for consumption by said patient.
21. Use of the food grade thickener according to any one of paragraphs 10 to 14 in the manufacture of a medicament for overcoming or ameliorating difficulties in swallowing in a patient in need of such treatment.
22. A storage and delivery system for a food grade thickener, comprising: a container containing the food grade thickener according to any one of paragraphs 10 to 14, and a pump dispenser sealingly attached to the container, said dispenser comprising a valve for inhibiting or preventing drying of the composition in the container.
23. A kit for a storage and delivery system for a food grade thickener, comprising: a container containing the food grade thickener according to any one of paragraphs 10 to 14, and a pump dispenser for attachment to the container, wherein said pump dispenser comprises a valve for inhibiting or preventing drying of the composition in the container.
24. A method of delivering a food grade thickener to an aqueous liquid or aqueous liquid solid mixture foodstuff, the method comprising the steps of:
providing a container containing the food grade thickener according to any one of paragraphs to 14, and applying a force to the pump dispenser to thereby deliver one or more doses of a predetermined volume of the food grade thickener to the foodstuff.
25 The system of paragraph 22, or the kit of paragraph 23, or the method of paragraph 24, wherein one, two and three doses of a predetermined volume of the food grade thickener increases the viscosity of said foodstuff to first, second and third viscosity levels respectively and wherein a nonlinear relationship exists between the first, second and third viscosity levels.
26. The system of paragraph 22 or 25, or the kit of paragraph 23 or paragraph 25, or the method of paragraph 24 or 25, wherein the pump dispenser comprises a valve for inhibiting or preventing drying of the composition in the container.
27. The system of any one of paragraphs 22 or 25-26, or the kit of any one of paragraphs 23 or 25-26, or the method of any one of paragraphs 24-26, wherein the valve is or comprises a self-sealing valve.
28. The system of paragraph 27, or the kit of paragraph 27, or the method of paragraph 27, wherein the valve is selected from the group consisting of a cross-slit valve, a ball valve, a flapper valve, an umbrella valve, a duck bill valve, a reed valve and any combination thereof.
29. The system of paragraph 27, or the kit of paragraph 27, or the method of paragraph 27, wherein the valve is biased to a closed position and is actuated to an open position upon application of a force to the pump dispenser forcing said composition to flow through the valve.
30. The system of any one of paragraphs 22 or 25-29, or the kit of any one of paragraphs 23 or 25-29, or the method of any one of paragraphs 24-29, wherein the pump dispenser comprises a dispenser tip, the dispenser tip including the valve disposed therein.
31. A swallowing disorder assisting or swallowing disorder ameliorating composition comprising a pourable, food grade thickener, having an apparent viscosity of about less than about 5,000 cPs measured at 20° C. using a #3 spindle at 5 rpm, and a resistance to flow of greater than about 12cm at 20° C. at 30 seconds measured using a Bostwick consistometer and wherein a 7 wt % solution of the food grade thickener and water has a transmittance of >90% at 650nm when measured with a 1 cm path length.
32. A method for providing a food grade thickener, the method comprising the steps of: establishing an aqueous continuous phase of a first polysaccharide, adding a second polysaccharide to the continuous phase thereby forming a gelled mixture, hydrolysing the gelled mixture to reduce the viscosity of the gelled mixture, and adding a gum to the hydrolysed gelled mixture under conditions such that the gum only partially expresses its viscosity, thereby forming the food grade thickener.
33. The method according to paragraph 32, wherein the first polysaccharide is selected from the group consisting of: agar, alginic acid, carrageenan, guar gum, gum tragacanth, gum ghatti, microcrystalline cellulose, sodium carboxymethylcellulose, methyl cellulose, hydroxypropylmethylcellulose, hydroxyproylcellulose, methylethylcellulose, gum karaya, locust bean gum, tara gum, psyllium seed gum, quince seed gum, a pectin, furcellaran, gellan gum, konjac, sodium alginate or xanthan gum and any combination thereof.
34. The method according to any one of paragraphs 32-33, wherein the aqueous continuous phase comprises between about 0.002 to 1.0 wt. % of the first polysaccharide.
35. The method according to any one of paragraphs 32-34, wherein the aqueous continuous phase is heated to melt the first polysaccharide.
36. The method according to any one of paragraphs 32-35, wherein the second polysaccharide is selected from the group consisting of: agar, alginic acid, carrageenan, guar gum, gum tragacanth, gum ghatti, microcrystalline cellulose, sodium carboxymethylcellulose, methyl cellulose, hydroxypropylmethylcellulose, hydroxyproylcellulose, methylethylcellulose, gum karaya, locust bean gum, tara gum, psyllium seed gum, quince seed gum, a pectin, furcellaran, gellan gum, konjac, sodium alginate, xanthan gum or a combination thereof.
37. The according to any one of paragraphs 32-36, wherein the second polysaccharide is added in a concentration of about 0.5 to 30 wt % to the aqueous phase.
38. The method according to any one of paragraphs 32-37, wherein the gelled mixture is hydrolysed at a temperature of about 50 to 95° C.
39. The method according to any one of paragraphs 32-38, wherein the gelled mixture is hydrolysed for a duration of about 2 to 72 hours.
40. The method according to any one of paragraphs 32-39, wherein the gelled mixture is acid hydrolysed to produce a hydrolysed gelled mixture.
41. The method according to any one of paragraphs 32-40, wherein the hydrolysed gelled mixture has a viscosity of between about 40-150 cP measured at 20° C. using a Brookfield viscometer #1 spindle at 10 RPM.
42. The method according to any one of paragraphs 32-41, wherein the gum is selected from the group consisting of: agar, alginic acid, carrageenan, guar gum, gum tragacanth, gum ghatti, microcrystalline cellulose, sodium carboxymethylcellulose, methyl cellulose, hydroxypropylmethylcellulose, hydroxyproylcellulose, methylethylcellulose, gum karaya, locust bean gum, tara gum, psyllium seed gum, quince seed gum, a pectin, furcellaran, gellan gum, konjac, sodium alginate, fenugreek gum, guar gum, tara gum and locust bean gum, xanthan gum and any combination thereof.
43. The method according to any one of paragraphs 32-42, wherein the gum is added in a concentration of about 2 to 30 wt %.

1. A method for providing a food grade thickener, the method comprising the steps of:
providing an aqueous phase,
adding a polysaccharide to the aqueous phase thereby forming a gelled mixture, hydrolysing the gelled mixture to reduce the viscosity of the gelled mixture, and adding a gum to the hydrolysed gelled mixture under conditions such that the gum only partially expresses its viscosity, thereby forming the food grade thickener.

2. The method according to claim 1, wherein the polysaccharide is selected from the group consisting of: agar, alginic acid, carrageenan, guar gum, gum tragacanth, gum ghatti, microcrystalline cellulose, sodium carboxymethylcellulose, methyl cellulose, hydroxypropylmethylcellulose, hydroxyproylcellulose, methylethylcellulose, gum karaya, locust bean gum, tara gum, psyllium seed gum, quince seed gum, a pectin, furcellaran, gellan gum, konjac, sodium alginate, xanthan gum or a combination thereof.

3. The method according to any one of the preceding claims, wherein the polysaccharide is added to the aqueous phase in a concentration of about 0.5 to 30 wt %.

4. The method according to any one of the preceding claims, wherein the gelled mixture is hydrolysed at a temperature of about 50 to 95° C.

5. The method according to any one of the preceding claims, wherein the gelled mixture is hydrolysed for a duration of about 2 to 72 hours.

6. The method according to any one of the preceding claims, wherein the gelled mixture is acid hydrolysed to produce the hydrolysed gelled mixture.

7. The method according to any one of the preceding claims, wherein the hydrolysed gelled mixture has a viscosity of between about 40-150 cP measured at 20° C. using a Brookfield viscometer #1 spindle at 10 rpm.

8. The method according to any one of the preceding claims, wherein the gum is selected from the group consisting of: agar, alginic acid, carrageenan, guar gum, gum tragacanth, gum ghatti, microcrystalline cellulose, sodium carboxymethylcellulose, methyl cellulose, hydroxypropylmethylcellulose, hydroxyproylcellulose, methylethylcellulose, gum karaya, locust bean gum, tara gum, psyllium seed gum, quince seed gum, a pectin, furcellaran, gellan gum, konjac, sodium alginate, fenugreek gum, guar gum, tara gum and locust bean gum, xanthan gum and any combination thereof.

9. The method according to any one of the preceding claims, wherein the gum is added in a concentration of about 2 to 30 wt %. A food grade thickener when produced by the method according to any one of claims 1 to 9.

11. The food grade thickener according to claim 10, wherein the food grade thickener is stable for at least six months.

12. The food grade thickener to any one of claims 10 to 11, wherein the food grade thickener has a viscosity of about 500 to 10,000 cP measured at 20° C. using a Brookfield viscometer #5 spindle at 10 rpm.

13. The food grade thickener to any one of claims 10 to 12, wherein the food grade thickener has a resistance to flow of greater than about 12 cm at 20° C. at 30 seconds measured using a Bostwick consistometer.

14. The food grade thickener to any one of claims 10 to 13, wherein a 7 wt % solution of the food grade thickener and water has a transmittance of about >90% at 650 nm when measured using a 1 cm path length.

15. A method for increasing the viscosity of an aqueous liquid or aqueous liquid solid mixture foodstuff, the method comprising the step of adding to the foodstuff the food grade thickener according to any one of claims 10 to 14.

16. The method according to claim 15, wherein the amount of food grade thickener that is added is about 1 to 30 wt %.

17. The method according to claim 15 or claim 16, wherein adding the food grade thickener to the foodstuff causes the viscosity of the foodstuff is increased to at least about 95 cP.

18. A method of treating a subject suffering from a mastication and/or deglutition disease, disorder or condition, the method comprising the step of administering a foodstuff to the subject, wherein the foodstuff comprises the food grade thickener according to any one of claims 10 to 14.

19. Use of the food grade thickener according to any one of claims 10 to 14 in the manufacture of a medicament for the treatment or amelioration of a mastication and/or deglutition disease, disorder or condition.

20. A method of overcoming or ameliorating difficulties in swallowing in a patient in need of such treatment, comprising the step of thickening a food or beverage with the food grade thickener according to any one of claims 10 to 14 for consumption by said patient

21. Use of the food grade thickener according to any one of claims 10 to 14 in the manufacture of a medicament for overcoming or ameliorating difficulties in swallowing in a patient in need of such treatment.

22. A storage and delivery system for a food grade thickener, comprising:
a container containing the food grade thickener according to any one of claims 10 to 14, and
a pump dispenser sealingly attached to the container, said dispenser comprising a valve for inhibiting or preventing drying of the composition in the container.

23. A kit for a storage and delivery system for a food grade thickener, comprising:
a container containing the food grade thickener according to any one of claims 10 to 14, and
a pump dispenser for attachment to the container, wherein said pump dispenser comprises a valve for inhibiting or preventing drying of the composition in the container.

24. A method of delivering a food grade thickener to an aqueous liquid or aqueous liquid solid mixture foodstuff, the method comprising the steps of:
providing a container containing the food grade thickener according to any one of claims 10 to 14, and
applying a force to the pump dispenser to thereby deliver one or more doses of a predetermined volume of the food grade thickener to the foodstuff.

25. The system of claim 22, or the kit of claim 23, or the method of claim 24, wherein one, two and three doses of a predetermined volume of the food grade thickener increases the viscosity of said foodstuff to first, second and third viscosity levels respectively and wherein a nonlinear relationship exists between the first, second and third viscosity levels.

26. The system of claim 22 or 25, or the kit of claim 23 or claim 25, or the method of claim 24 or 25, wherein the pump dispenser comprises a valve for inhibiting or preventing drying of the composition in the container.

27. The system of any one of claim 22 or 25-26, or the kit of any one of claim 23 or 25-26, or the method of any one of claims 24-26, wherein the valve is or comprises a self-sealing valve.

28. The system of claim 27, or the kit of claim 27, or the method of claim 27, wherein the valve is selected from the group consisting of a cross-slit valve, a ball valve, a flapper valve, an umbrella valve, a duck bill valve, a reed valve and any combination thereof.

29. The system of claim 27, or the kit of claim 27, or the method of claim 27, wherein the valve is biased to a closed position and is actuated to an open position upon application of a force to the pump dispenser forcing said composition to flow through the valve.

30. The system of any one of claim 22 or 25-29, or the kit of any one of claim 23 or 25-29, or the method of any one of claims 24-29, wherein the pump dispenser comprises a dispenser tip, the dispenser tip including the valve disposed therein.

31. A swallowing disorder assisting or swallowing disorder ameliorating composition comprising a pourable, food grade thickener, having an apparent viscosity of about less than about 5,000 cPs measured at 20° C. using a #3 spindle at 5 rpm, and a resistance to flow of greater than about 12 cm at 20° C. at 30 seconds measured using a Bostwick consistometer and wherein a 7 wt % solution of the food grade thickener and water has a transmittance of >90% at 650 nm when measured with a 1 cm path length.

32. A method for providing a food grade thickener, the method comprising the steps of:
establishing an aqueous continuous phase of a first polysaccharide,
adding a second polysaccharide to the continuous phase thereby forming a gelled mixture,
hydrolysing the gelled mixture to reduce the viscosity of the gelled mixture, and adding a gum to the hydrolysed gelled mixture under conditions such that the gum only partially expresses its viscosity, thereby forming the food grade thickener.

33. The method according to claim 32, wherein the first polysaccharide is selected from the group consisting of: agar, alginic acid, carrageenan, guar gum, gum tragacanth, gum ghatti, microcrystalline cellulose, sodium carboxymethylcellulose, methyl cellulose, hydroxypropylmethylcellulose, hydroxyproylcellulose, methylethylcellulose, gum karaya, locust bean gum, tara gum, psyllium seed gum, quince seed gum, a pectin, furcellaran, gellan gum, konjac, sodium alginate or xanthan gum and any combination thereof.

34. The method according to any one of claims 32-33, wherein the aqueous continuous phase comprises between about 0.002 to 1.0 wt. % of the first polysaccharide.

35. The method according to any one of claims 32-34, wherein the aqueous continuous phase is heated to melt the first polysaccharide.

36. The method according to any one of claims 32-35, wherein the second polysaccharide is selected from the group consisting of: agar, alginic acid, carrageenan, guar gum, gum tragacanth, gum ghatti, microcrystalline cellulose, sodium carboxymethylcellulose, methyl cellulose, hydroxypropylmethylcellulose, hydroxyproylcellulose, methylethylcellulose, gum karaya, locust bean gum, tara gum, psyllium seed gum, quince seed gum, a pectin, furcellaran, gellan gum, konjac, sodium alginate, xanthan gum or a combination thereof.

37. The according to any one of claims 32-36, wherein the second polysaccharide is added in a concentration of about 0.5 to 30 wt % to the aqueous phase.

38. The method according to any one of claims 32-37, wherein the gelled mixture is hydrolysed at a temperature of about 50 to 95° C.

39. The method according to any one of claims 32-38, wherein the gelled mixture is hydrolysed for a duration of about 2 to 72 hours.

40. The method according to any one of claims 32-39, wherein the gelled mixture is acid hydrolysed to produce a hydrolysed gelled mixture.

41. The method according to any one of claims 32-40, wherein the hydrolysed gelled mixture has a viscosity of between about 40-150 cP measured at 20° C. using a Brookfield viscometer #1 spindle at 10 rpm.

42. The method according to any one of claims 32-41, wherein the gum is selected from the group consisting of: agar, alginic acid, carrageenan, guar gum, gum tragacanth, gum ghatti, microcrystalline cellulose, sodium carboxymethylcellulose, methyl cellulose, hydroxypropylmethylcellulose, hydroxyproylcellulose, methylethylcellulose, gum karaya, locust bean gum, tara gum, psyllium seed gum, quince seed gum, a pectin, furcellaran, gellan gum, konjac, sodium alginate, fenugreek gum, guar gum, tara gum and locust bean gum, xanthan gum and any combination thereof.

43. The method according to any one of claims 32-42, wherein the gum is added in a concentration of about 2 to 30 wt %.
